SEQUENCE LISTING (37 sequences)

SEQ ID NO: 1 - protein, 1273 aa, Severe acute respiratory syndrome coronavirus 2
  MISC_FEATURE (1)..(1273): Spike protein of SARS-Co-V2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 150 aa, Artificial Sequence
  Synthetic- C7L polypeptide
MGIQHEFDIIINGDIALRNLQLHKGDNYGCKLKIISNDYKKLKFRFIIRPDWSEIDEVKGLTVFANNYAVKVNKVDDTFYYVIYEAVIHLYNKKTEILIYSDDENELFKHYYPYISLNMISKKYKVKEENYSSPYIEHPLIPYRDYESMD

SEQ ID NO: 3 - protein, 284 aa, Artificial Sequence
  Synthetic- K1L polypeptide
MDLSRINTWKSKQLKSFLSSKDTFKADVHGHSALYYAIADNNVRLVCTLLNAGALKNLLENEFPLHQAATLEDTKIVKILLFSGMDDSQFDDKGNTALYYAVDSGNMQTVKLFVKKNWRLMFYGKTGWKTSFYHAVMLNDVSIVSYFLSEIPSTFDLAILLSCIHTTIKNGHVDMMILLLDYMTSTNTNNSLLFIPDIKLAIDNKDIEMLQALFKYDINIYSVNLENVLLDDAEITKMIIEKHVEYKSDSYTKDLDIVKNNKLDEIISKNKELRLMYVNCVKKN

SEQ ID NO: 4 - DNA, 11 bp, Artificial Sequence
  Synthetic- TEE
aaaactgctaa

SEQ ID NO: 5 - DNA, 26 bp, Artificial Sequence
  Synthetic- Viral Synthetic Late Promoter (SLP)
catattgaagagacagagtgatatat

SEQ ID NO: 6 - protein, 193 aa, Artificial Sequence
  Synthetic- SARS-CoV-2 receptor binding domain (RBD)
FRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 7 - protein, 222 aa, Artificial Sequence
  Synthetic- M protein of SARS-CoV-2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 8 - protein, 75 aa, Artificial Sequence
  Synthetic- E protein of SARS-Co-V-2
MYSFVSEETGTLIVNSVLLFLAFVVFLLVTLAILTALRLCAYCCNIVNVSLVKPSFYVYSRVKNLNSSRVPDLLV

SEQ ID NO: 9 - protein, 419 aa, Artificial Sequence
  Synthetic- N protein of SARS-CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 10 - DNA, 37 bp, Artificial Sequence
  Synthetic- TEE
catattgaagagacagagtgatatataaaactgctaa

SEQ ID NO: 11 - DNA, 42 bp, Artificial Sequence
  Synthetic- TEE
agaaccatattgaagagacagagtgatatataaaactgctaa

SEQ ID NO: 12 - DNA, 90 bp, Artificial Sequence
  Synthetic- TEE
agaaccatattgaagagacagagtgatatataaaactgctaactcaagcagcacaagaattaaatgaataccaagaaaatacttggccag

SEQ ID NO: 13 - protein, 9 aa, Artificial Sequence
  Synthetic- ebola glycoprotein epitope 6D8 tag
VYKLDISEA

SEQ ID NO: 14 - DNA, 5098 bp, Artificial Sequence
  Synthetic- SARS-CoV2- spike flanked by TK recombination arms
  misc_feature (11)..(691): TK right arm CpVV
  misc_feature (11)..(593): Right arm TK
  misc_feature (715)..(756): E/L Promoter
  misc_feature (768)..(4592): Spike
  misc_feature (775)..(784): Trx Term
  misc_feature (3040)..(3048): Trx Term
  misc_feature (3888)..(3898): Trx Term
  misc_feature (4092)..(4100): Trx Term
  misc_feature (4591)..(5090): Left arm TK
ggcgcgccctgaatatgaaggagcaaaaggttgtaacattttattaccgtgtgggatataaaagtccttgatccattgatctggaaacgggcatctccatttaagactagatgccacggggtttaaaatactaatcatgacattttgtagagcgtaattacttagtaaatccgccgtactaggttcatttcctcctcgtttggatcttacatcagaaattaaaataatcttagaaggatgcagttgttttttgatggatcgtagatattcctcatcaacgaaccgagtcactagagtcacatcacgcaatccatttagaataggatcatgatggcgtccgtcaattagcatccatttgataatcattcctaaattatagaaatgatctctcaaataacgtatatgtgtaccaggagccgatcctatatacactacggtggcaccatctaatataccgtgtcgctgtaacttactaagaaaaaataattctcctagtaatagttttaactgtccttgatacggcagtttttttgcgacctcatttgcactttctggttcgtaatctaactcattatcaatttcctcaaaatacataaacggtttatctaacgacacaacatccatttttaagtattatattaaaatttaatcaatgtttatttttagttttttagataaaaaatataatattagttaattaggcggccgcgtcgacaaaaattgaaattttattttttttttttggaatataaatagactcgaggccaccatgtttgtgtttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcttaccatttaatgatggtgtttattttgcttccactgagaagtctaacataataagaggctggatttttggtactactttagattcgaagacccagtccttattaattgttaataacgctactaatgttgttattaaagtctgtgaatttcaattttgtaatgatccatttttaggtgtttattaccacaaaaacaacaaaagttggatggaaagtgagttcagagtttattctagtgcgaataattgcacttttgaatatgtctctcagccttttttaatggaccttgaaggaaaacagggtaatttcaaaaatttaagagaatttgtgtttaagaatattgatggttattttaaaatatattctaagcacacgcctattaatttagtgagagatctccctcagggtttttcggctttagaaccattggtagatttaccaataggtattaacatcactagatttcaaactttattagctttacatagaagttatttaactcctggtgattcttcttcaggttggacagctggtgctgcagcttattatgtgggttatttacaacctagaactttcttattaaaatataatgaaaatggaaccattacagatgctgtagactgtgcattagaccctttatcagaaacaaagtgtacgttaaaatccttcactgtagaaaaaggaatctatcaaacttctaactttagagtccaaccaacagaatctattgttagatttcctaatattacaaacttatgcccttttggtgaagtttttaacgccaccagatttgcatctgtttatgcttggaacagaaagagaatcagcaactgtgttgctgattattctgtcttatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatttagattctaaggttggtggtaattataattacttatatagattatttagaaagtctaatttaaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtattatcttttgaacttttacatgcaccagcaactgtttgtggacctaaaaagtctactaatttagttaaaaacaaatgtgtcaatttcaacttcaatggtttaacaggcacaggtgttttaactgagtctaacaaaaagtttctgcctttccaacaatttggcagagacattgctgacactactgatgctgtcagagatccacagacattagagattttagacattacaccatgttcttttggtggtgtcagtgttataacaccaggaacaaatacttctaaccaggttgctgttttatatcaggatgttaactgcacagaagtccctgttgctattcatgcagatcaattaactcctacttggagagtttattctacaggttctaatgtttttcaaacaagagcaggctgtttaataggggctgaacatgtcaacaactcatatgagtgtgacatacccattggtgcaggtatatgcgctagttatcagactcagactaattctcctgggagcgcaagtagtgtagctagtcaatccatcattgcctacactatgtcattaggtgcagaaaattcagttgcttactctaataactctattgccatacccacaaattttactattagtgttaccacagaaattttaccagtgtctatgaccaagacatcagtagattgtacaatgtacatttgtggtgattcaactgaatgcagcaatcttttgttgcaatatggcagtttctgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagagatcaccaattgaagatttacttttcaacaaagtgacattagcagatgctggcttcatcaaacaatatggtgattgcttaggtgatattgctgctagagacttaatttgtgcacaaaagtttaacggcttaactgttttaccacctttattaacagatgaaatgattgctcaatacacttctgcattattagcgggtacaatcacttctggttggacctttggtgcaggtccggcattacaaataccatttccgatgcaaatggcttatagatttaatggtattggagttacacagaatgttttatatgagaaccaaaaattaattgccaaccaatttaatagtgctattggcaaaattcaagactcattatcttccacaccaagtgcattaggaaaattacaagatgtggtcaaccaaaatgcacaagctttaaacacgttagttaaacaattaagctccaattttggtgcaatttcaagtgttttaaatgatatcttatcaagattagacccaccagaggctgaagtgcaaattgatagattaatcacaggcagattacaaagtttacagacatatgtgactcaacaattaattagagctgcagaaatcagagcttctgctaatttagctgctactaaaatgtcagagtgtgtattaggacaatcaaaaagagttgatttctgtggaaagggctatcatttaatgtccttccctcagtcagcacctcatggtgtagtcttcttacatgtgacttatgtccctgcacaagaaaagaacttcacaactgctcctgccatttgtcatgatggaaaagcacactttcctagagaaggtgtctttgtttcaaatggcacacactggtttgtaacacaaagaaatttctatgaaccacaaatcattactacagacaacacatttgtgtctggtaactgtgatgttgtaataggaattgtcaacaacacagtttatgatcctttacaacctgaattagactcattcaaggaggagttagataaatattttaagaatcatacatcaccagatgttgatttaggtgacatctctggcattaatgcttcagttgtaaacattcaaaaagaaattgacagattaaatgaggttgccaagaatttaaatgaatctttaatcgatttacaagaattaggaaagtatgagcagtatataaaatggccatggtacatttggttaggttttatagctggcttaattgccatagtaatggtgacaattatgttatgctgtatgaccagttgctgtagttgtttaaagggctgttgttcttgtggatcctgctgcaaatttgatgaagacgactctgagccagtgttaaaaggagtcaaattacattacacataaccgtcagtatcggcggaattacagctgagcgccggtcgctaccattaccagttggtctggtgtcaaaaataatttttctcccgggtagctagttaattacatgatgacaataaagaattaattattgttcactttattcgactttaatatatccatcacgttagaaaatgcgatatcgcgacgaggatctatgtatctaataggatctattgcggtggtagctagagaggattcttttttgaatcgcatcaaactaatcacaaagtcgaacaaatatcctttattaagtttgacccttccatctgtaacaatagggaccttgttaaacagttttttaaaatcttgagagtctgtgaattttgtcaattgtctgtattcctctgaaagagattcataacaatgacccacggcttctaatttattttttgattggatcaataataataacagaaagtctagatattgagtgatttgcaatatatcagataatgaagattcacctcgagg

SEQ ID NO: 15 - protein, 1273 aa, Artificial Sequence; identical to SEQ ID NO: 1
  Synthetic- modified S protein SARS-CoV-2
  MISC_FEATURE (682)..(685): furin cleavage site
  MISC_FEATURE (817)..(817): F817P
  MISC_FEATURE (892)..(892): A892P
  MISC_FEATURE (899)..(899): A899P
  MISC_FEATURE (942)..(942): A942P
  MISC_FEATURE (986)..(986): K986P
  MISC_FEATURE (987)..(987): V987P

SEQ ID NO: 16 - DNA, 5098 bp, Severe acute respiratory syndrome coronavirus 2
  misc_feature (1)..(5098): SARS-CoV-2 Spike sequence
ggcgcgccctgaatatgaaggagcaaaaggttgtaacattttattaccgtgtgggatataaaagtccttgatccattgatctggaaacgggcatctccatttaagactagatgccacggggtttaaaatactaatcatgacattttgtagagcgtaattacttagtaaatccgccgtactaggttcatttcctcctcgtttggatcttacatcagaaattaaaataatcttagaaggatgcagttgttttttgatggatcgtagatattcctcatcaacgaaccgagtcactagagtcacatcacgcaatccatttagaataggatcatgatggcgtccgtcaattagcatccatttgataatcattcctaaattatagaaatgatctctcaaataacgtatatgtgtaccaggagccgatcctatatacactacggtggcaccatctaatataccgtgtcgctgtaacttactaagaaaaaataattctcctagtaatagttttaactgtccttgatacggcagtttttttgcgacctcatttgcactttctggttcgtaatctaactcattatcaatttcctcaaaatacataaacggtttatctaacgacacaacatccatttttaagtattatattaaaatttaatcaatgtttatttttagttttttagataaaaaatataatattagttaattaggcggccgcgtcgacaaaaattgaaattttattttttttttttggaatataaatagactcgaggccaccatgtttgtgtttcttgttttattaccattagtctctagtcagtgtgttaatttaacaaccagaactcaattaccccctgcatacactaattctttcacaagaggtgtttattaccctgacaaagttttcagatcctcagttttacattcaactcaggacttattcttacctttcttttccaatgttacttggttccatgctatacatgtctctgggaccaatggtactaagaggtttgataaccctgtcttaccatttaatgatggtgtttattttgcttccactgagaagtctaacataataagaggctggatttttggtactactttagattcgaagacccagtccttattaattgttaataacgctactaatgttgttattaaagtctgtgaatttcaattttgtaatgatccatttttaggtgtttattaccacaaaaacaacaaaagttggatggaaagtgagttcagagtttattctagtgcgaataattgcacttttgaatatgtctctcagccttttttaatggaccttgaaggaaaacagggtaatttcaaaaatttaagagaatttgtgtttaagaatattgatggttattttaaaatatattctaagcacacgcctattaatttagtgagagatctccctcagggtttttcggctttagaaccattggtagatttaccaataggtattaacatcactagatttcaaactttattagctttacatagaagttatttaactcctggtgattcttcttcaggttggacagctggtgctgcagcttattatgtgggttatttacaacctagaactttcttattaaaatataatgaaaatggaaccattacagatgctgtagactgtgcattagaccctttatcagaaacaaagtgtacgttaaaatccttcactgtagaaaaaggaatctatcaaacttctaactttagagtccaaccaacagaatctattgttagatttcctaatattacaaacttatgcccttttggtgaagtttttaacgccaccagatttgcatctgtttatgcttggaacagaaagagaatcagcaactgtgttgctgattattctgtcttatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatttagattctaaggttggtggtaattataattacttatatagattatttagaaagtctaatttaaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtattatcttttgaacttttacatgcaccagcaactgtttgtggacctaaaaagtctactaatttagttaaaaacaaatgtgtcaatttcaacttcaatggtttaacaggcacaggtgttttaactgagtctaacaaaaagtttctgcctttccaacaatttggcagagacattgctgacactactgatgctgtcagagatccacagacattagagattttagacattacaccatgttcttttggtggtgtcagtgttataacaccaggaacaaatacttctaaccaggttgctgttttatatcaggatgttaactgcacagaagtccctgttgctattcatgcagatcaattaactcctacttggagagtttattctacaggttctaatgtttttcaaacaagagcaggctgtttaataggggctgaacatgtcaacaactcatatgagtgtgacatacccattggtgcaggtatatgcgctagttatcagactcagactaattctcctgggagcgcaagtagtgtagctagtcaatccatcattgcctacactatgtcattaggtgcagaaaattcagttgcttactctaataactctattgccatacccacaaattttactattagtgttaccacagaaattttaccagtgtctatgaccaagacatcagtagattgtacaatgtacatttgtggtgattcaactgaatgcagcaatcttttgttgcaatatggcagtttctgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagagatcaccaattgaagatttacttttcaacaaagtgacattagcagatgctggcttcatcaaacaatatggtgattgcttaggtgatattgctgctagagacttaatttgtgcacaaaagtttaacggcttaactgttttaccacctttattaacagatgaaatgattgctcaatacacttctgcattattagcgggtacaatcacttctggttggacctttggtgcaggtccggcattacaaataccatttccgatgcaaatggcttatagatttaatggtattggagttacacagaatgttttatatgagaaccaaaaattaattgccaaccaatttaatagtgctattggcaaaattcaagactcattatcttccacaccaagtgcattaggaaaattacaagatgtggtcaaccaaaatgcacaagctttaaacacgttagttaaacaattaagctccaattttggtgcaatttcaagtgttttaaatgatatcttatcaagattagacccaccagaggctgaagtgcaaattgatagattaatcacaggcagattacaaagtttacagacatatgtgactcaacaattaattagagctgcagaaatcagagcttctgctaatttagctgctactaaaatgtcagagtgtgtattaggacaatcaaaaagagttgatttctgtggaaagggctatcatttaatgtccttccctcagtcagcacctcatggtgtagtcttcttacatgtgacttatgtccctgcacaagaaaagaacttcacaactgctcctgccatttgtcatgatggaaaagcacactttcctagagaaggtgtctttgtttcaaatggcacacactggtttgtaacacaaagaaatttctatgaaccacaaatcattactacagacaacacatttgtgtctggtaactgtgatgttgtaataggaattgtcaacaacacagtttatgatcctttacaacctgaattagactcattcaaggaggagttagataaatattttaagaatcatacatcaccagatgttgatttaggtgacatctctggcattaatgcttcagttgtaaacattcaaaaagaaattgacagattaaatgaggttgccaagaatttaaatgaatctttaatcgatttacaagaattaggaaagtatgagcagtatataaaatggccatggtacatttggttaggttttatagctggcttaattgccatagtaatggtgacaattatgttatgctgtatgaccagttgctgtagttgtttaaagggctgttgttcttgtggatcctgctgcaaatttgatgaagacgactctgagccagtgttaaaaggagtcaaattacattacacataaccgtcagtatcggcggaattacagctgagcgccggtcgctaccattaccagttggtctggtgtcaaaaataatttttctcccgggtagctagttaattacatgatgacaataaagaattaattattgttcactttattcgactttaatatatccatcacgttagaaaatgcgatatcgcgacgaggatctatgtatctaataggatctattgcggtggtagctagagaggattcttttttgaatcgcatcaaactaatcacaaagtcgaacaaatatcctttattaagtttgacccttccatctgtaacaatagggaccttgttaaacagttttttaaaatcttgagagtctgtgaattttgtcaattgtctgtattcctctgaaagagattcataacaatgacccacggcttctaatttattttttgattggatcaataataataacagaaagtctagatattgagtgatttgcaatatatcagataatgaagattcacctcgagg

SEQ ID NO: 17 - protein, 1273 aa, Artificial Sequence; identical to SEQ ID NO: 1
  Synthetic- pfs-spike
  MISC_FEATURE (682)..(685): rrar mutation furin cleavage site
  MISC_FEATURE (817)..(817): F817P
  MISC_FEATURE (892)..(892): A892P
  MISC_FEATURE (899)..(899): A899P
  MISC_FEATURE (942)..(942): A942P
  MISC_FEATURE (986)..(986): K986P
  MISC_FEATURE (987)..(987): V987P

SEQ ID NO: 18 - protein, 190 aa, Artificial Sequence
  Synthetic- SARS-CoV-2 receptor binding domain (RBD) G339D
  MISC_FEATURE (19)..(19): G339D mutation
QPTESIVRFPNITNLCPFDEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 19 - protein, 190 aa, Artificial Sequence
  Synthetic- SARS-CoV-2 receptor binding domain (RBD) S373P
  MISC_FEATURE (51)..(51): S371L mutation
QPTESIVRFPNITNLCPFGEVFNATRFASVYAWNRKRISNCVADYSVLYNLASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 20 - protein, 190 aa, Artificial Sequence
  Synthetic- SARS-CoV-2 receptor binding domain (RBD) S373P
  MISC_FEATURE (53)..(53): S373P mutation
QPTESIVRFPNITNLCPFGEVFNATRFASVYAWNRKRISNCVADYSVLYNSAPFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 21 - protein, 190 aa, Artificial Sequence
  Synthetic- SARS-CoV-2 receptor binding domain (RBD) S375F
  MISC_FEATURE (55)..(55): S375F mutation
QPTESIVRFPNITNLCPFGEVFNATRFASVYAWNRKRISNCVADYSVLYNSASFFTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 22 - protein, 190 aa, Artificial Sequence
  Synthetic- SARS-CoV-2 receptor binding domain (RBD) K417N
  MISC_FEATURE (97)..(97): K417N mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 23 - protein, 190 aa, Artificial Sequence
  Synthetic- SARS-CoV-2 receptor binding domain (RBD) K417T
  MISC_FEATURE (97)..(97): K417N mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 24 - protein, 190 aa, Artificial Sequence
  Synthetic- SARS-CoV-2 receptor binding domain (RBD) K417M
  MISC_FEATURE (97)..(97): K417M mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGMIADYNYKLPDDFTGCVIAWNSNNLDSKVGGNYNYLYRLFRKSNLKPFERDISTEIYQAGSTPCNGVEGFNCYFPLQSYGFQPTNGVGYQPYRV

SEQ ID NO: 25 - protein, 190 aa, Artificial Sequence
  Synthetic- SARS-CoV-2 receptor binding domain (RBD) N440K
  MISC_FEATURE (120)..(120): N440K mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNKLDSKVGGNYNYLYRLFRKSNLKPFERDISTEIYQAGSTPCNGVEGFNCYFPLQSYGFQPTNGVGYQPYRV

SEQ ID NO: 26 - protein, 190 aa, Artificial Sequence
  Synthetic-  SARS-CoV-2 receptor binding domain (RBD) G446S
  MISC_FEATURE (126)..(126): G446S mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVSGNYNYLYRLFRKSNLKPFERDISTEIYQAGSTPCNGVEGFNCYFPLQSYGFQPTNGVGYQPYRV

SEQ ID NO: 27 - protein, 190 aa, Artificial Sequence
  Synthetic- SARS-CoV-2 receptor binding domain (RBD) L452R
  MISC_FEATURE (132)..(132): L542R mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRV

SEQ ID NO: 28 - protein, 190 aa, Artificial Sequence
  Synthetic- SARS-CoV-2 receptor binding domain (RBD) S477N
  MISC_FEATURE (157)..(157): S477N mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGNTPCNGVEGFNCYFPLQSYGFQPTNGVGYQPYRV

SEQ ID NO: 29 - protein, 190 aa, Artificial Sequence
  Synthetic- SARS-CoV-2 receptor binding domain (RBD) T478K
  MISC_FEATURE (158)..(158): T478K mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSKPCNGVEGFNCYFPLQSYGFQPTNGVGYQPYRV

SEQ ID NO: 30 - protein, 190 aa, Artificial Sequence
  Synthetic- SARS-CoV-2 receptor binding domain (RBD) E484K
  MISC_FEATURE (164)..(164): E484K mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVKGFNCYFPLQSYGFQPTNGVGYQPYRV

SEQ ID NO: 31 - protein, 190 aa, Artificial Sequence
  Synthetic- SARS-CoV-2 receptor binding domain (RBD) E484A
  MISC_FEATURE (164)..(164): E484A mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVAGFNCYFPLQSYGFQPTNGVGYQPYRV

SEQ ID NO: 32 - protein, 190 aa, Artificial Sequence
  Synthetic- SARS-CoV-2 receptor binding domain (RBD) Q493K
  MISC_FEATURE (173)..(173): Q493K mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLKSYGFQPTNGVGYQPYRV

SEQ ID NO: 33 - protein, 190 aa, Artificial Sequence
  Synthetic- SARS-CoV-2 receptor binding domain (RBD) Q493R
  MISC_FEATURE (173)..(173): Q493R mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLRSYGFQPTNGVGYQPYRV

SEQ ID NO: 34 - protein, 190 aa, Artificial Sequence
  Synthetic- SARS-CoV-2 receptor binding domain (RBD) G496S
  MISC_FEATURE (176)..(176): G496S mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYSFQPTNGVGYQPYRV

SEQ ID NO: 35 - protein, 190 aa, Artificial Sequence
  Synthetic- SARS-CoV-2 receptor binding domain (RBD) Q498R
  MISC_FEATURE (178)..(178): Q498R mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFRPTNGVGYQPYRV

SEQ ID NO: 36 - protein, 190 aa, Artificial Sequence
  Synthetic- SARS-CoV-2 receptor binding domain (RBD) N501Y
  MISC_FEATURE (181)..(181): N501Y mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRV

SEQ ID NO: 37 - protein, 190 aa, Artificial Sequence
  Synthetic- SARS-CoV-2 receptor binding domain (RBD) Y505H
  MISC_FEATURE (185)..(185): Y505H mutation
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGHQPYRV

(19) United States
(12) Patent Application Publication
(10) Pub. No.: US 2024/0226273 A1
(43) Pub. Date: Jul. 11, 2024

(54) Highly Attenuated Replication-Competent Recombinant Poxvirus As A Vaccine Platform And Methods Of Use

(71) Applicant: Arizona Board of Regents on behalf of Arizona State University, Scottsdale, AZ (US)

(72) Inventors: Bertram Jacobs, Tempe, AZ (US); Karen Kibler, Scottsdale, AZ (US)

(73) Assignee: Arizona Board of Regents on behalf of Arizona State University, Scottsdale, AZ (US)

(21) Appl. No.: 18/283,042

(22) PCT Filed: Mar. 30, 2022
(86) PCT No.: PCT/US2022/022541

§ 371 Date: Sep. 20, 2023

Related U.S. Application Data
(60) Provisional application No. 63/168,140, filed on Mar. 30, 2021.
(60) Provisional application No. 63/251,319, filed on Oct. 1, 2021.
(60) Provisional application No. 63/286,961, filed on Dec. 7, 2021.

(51) Int. Cl. A61K 39/215; A61K 39/00; A61P 31/14; C12N 15/86

(52) U.S. Cl. CPC A61K 39/215; A61P 31/14; C12N 15/86; A61K 2039/5256; A61K 2039/543; C12N 2710/24143; C12N 2770/20022; C12N 2770/20034; C12N 2840/105; C12N 2840/206

(57) ABSTRACT

Recombinant poxvirus expressing severe acute respiratory syndrome coronavirus 2 structural proteins and virus-like particles are described, along with methods of making and using the same.

### CROSS-REFERENCE TO RELATED APPLICATIONS

This patent application claims the benefit of priority of United States Provisional Patent Application Nos. 63/168,140 filed on Mar. 30, 2021, 63/251,319 filed Oct. 1, 2021 and 63/286,961 filed on Dec. 7, 2021, which are all incorporated herein by reference in their entirety.

### STATEMENT REGARDING FEDERALLY SPONSORED RESEARCH

This invention was made with government support from the National Institutes of Health under grant numbers P01 AI060699 and ROI AI129269. The government has certain rights in the invention.

### SEQUENCE LISTING

This application is being filed electronically via EFS-Web and includes an electronically submitted Sequence Listing in .txt format. The .txt file contains a sequence listing entitled “112624_01328_ST25.txt” created on Mar. 30, 2022 and is 99, 321 bytes in size. The Sequence Listing contained in this .txt file is part of the specification and is hereby incorporated by reference herein in its entirety.

### BACKGROUND

Coronaviruses (CoV) constitute a large family of positive-stranded, enveloped RNA viruses that infect a broad range of mammalian and avian species. The viruses cause primarily respiratory and enteric diseases. In the last two decades three new zoonotic CoVs have emerged to infect humans. The most recent emergence of SARS-COV-2 that continues to spread globally raises many scientific and public health questions and challenges. Development of effective vaccines and antiviral therapeutics and rapid deployment of both is a pressing need. This will be an even more critical priority if SARS-COV-2 continues to spread and becomes endemic in the respiratory virus disease landscape. Previous work with the other two recent emergent pathogenic human CoVs, severe acute respiratory syndrome (SARS-COV) and Middle East respiratory syndrome (MERS-COV), provides insight and platforms that can help expedite the process, but none of these have moved beyond early trial stages. Much remains to be learned about the SARS-CoV-2 and its interplay with its human host and what will constitute the most effective, safe vaccine strategy. There are currently seven CoVs that infect humans, HCoVs OC43, 229E, NL63 and HKU1, that cause seasonal upper respiratory infections, in addition to the three more pathogenic viruses. The human viruses are thought to have emerged from zoonotic hosts to infect humans. Viral genomic analyses indicate that the human viruses are related to bat CoVs. A large number of novel CoVs have been identified in bat populations since identification of SARS-COV and the expectation is that we will continue to have spillover of these viruses to humans. This reinforces the need for development of vaccines against emergent CoVs.

### SUMMARY OF THE INVENTION

In a first aspect, provided herein is a recombinant NYVAC vector comprising a polynucleotide encoding a severe acute respiratory syndrome coronavirus 2 (SARS-COV-2) antigen; a polynucleotide encoding C7L (SEQ ID NO:2) adjacent to a polynucleotide encoding KIL (SEQ ID NO:3); and a translation enhancing element (TEE). In some embodiments, a promoter is operably connected to both a translation enhancing element (TEE) and a polynucleotide encoding a severe acute respiratory syndrome coronavirus 2 (SARS-COV-2) antigen. In some embodiments, the SARS-COV-2 antigen is selected from the group consisting of SARS-Cov-2 spike (S) protein (SEQ ID NO:1), SARS-COV-2 receptor binding domain (RBD) (SEQ ID NO:6), SARS-COV-2 membrane (M) protein (SEQ ID NO:7), SARS-COV-2 envelope (E) protein (SEQ ID NO:8), SARS-COV-2 nucleocapsid (N) protein (SEQ ID NO:9), pfs-spike (pre-fusion state spike) SARS-COV-2 (SEQ ID NO: 17), sequences at least 90% identical to any of the listed antigens, and combinations or fragments thereof. In some embodiments, the SARS-CoV-2 antigen is SARS-COV-2 S protein (SEQ ID NO:1) or a sequence at least 90% identical thereto.

In some embodiments, the vector comprises polynucleotides encoding at least two SARS-COV-2 antigens selected from the group consisting of SARS-Cov-2 spike (S) protein (SEQ ID NO:1), SARS-COV-2 receptor binding domain (RBD) (SEQ ID NO:6), SARS-COV-2 membrane (M) protein (SEQ ID NO:7), SARS-COV-2 envelope (E) protein (SEQ ID NO:8), SARS-COV-2 nucleocapsid (N) protein (SEQ ID NO:9), pfs-spike of SARS-COV-2 (a polynucleotide encoding SEQ ID NO:17), and sequences at least 90% identical to any of the listed polynucleotides. In some embodiments, the vector comprises polynucleotides encoding at least three SARS-COV-2 antigens selected from the group consisting of SARS-Cov-2 spike (S) protein (SEQ ID NO:1), SARS-COV-2 receptor binding domain (RBD) (SEQ ID NO:6), SARS-COV-2 membrane (M) protein (SEQ ID NO:7), SARS-COV-2 envelope (E) protein (SEQ ID NO:8), SARS-COV-2 nucleocapsid (N) protein (SEQ ID NO:9), pfs-spike SARS-COV-2 (SEQ ID NO: 17), and sequences at least 90% identical thereto. In some embodiments, the vector comprises polynucleotides encoding at least four SARS-COV-2 antigens selected from the group consisting of SARS-Cov-2 spike (S) protein (SEQ ID NO:1), SARS-COV-2 receptor binding domain (RBD) (SEQ ID NO:6), SARS-COV-2 membrane (M) protein (SEQ ID NO:7), SARS-COV-2 envelope (E) protein (SEQ ID NO:8), SARS-COV-2 nucleocapsid (N) protein (SEQ ID NO:9), pfs-spike SARS-COV-2 (SEQ ID NO: 17), and sequences at least 90% identical thereto.

In some embodiments, the translation enhancing element comprises SEQ ID NO:4. In some embodiments, the NYVAC vector additionally comprises a synthetic late promoter (SLP). In some embodiments, the SLP comprises SEQ ID NO:5. In some embodiments, the NYVAC vector additionally comprises an internal ribosomal entry site (IRES) to allow for expression of more than one antigenic polypeptide. In some embodiments, the NYVAC vector comprises at least two IRES. In some embodiments, the NYVAC vector additionally comprises a self-cleaving protein element. In some embodiments, the NYVAC comprises at least two self-cleaving protein elements.

In a second aspect, provided herein is a vaccine composition comprising a recombinant NYVAC vector as described herein and a pharmaceutically acceptable carrier. In some embodiments, the vaccine composition additionally comprises an adjuvant.

In a third aspect, provided herein is a method of inducing an immune response against a SARS-COV-2 antigen in a subject comprising administering an effective amount of a vaccine composition described herein to the subject. In some embodiments, the SARS-COV-2 antigen is selected from the group consisting of SARS-Cov-2 spike (S) protein (SEQ ID NO:1), SARS-COV-2 receptor binding domain (RBD) (SEQ ID NO:6), SARS-COV-2 membrane (M) protein (SEQ ID NO:7), SARS-COV-2 envelope (E) protein (SEQ ID NO:8), SARS-COV-2 nucleocapsid (N) protein (SEQ ID NO:9), pfs-spike SARS-COV-2 (SEQ ID NO: 17), sequences at least 90% identical thereto, and fragments or combinations thereof. In some embodiments, the SARS-COV-2 antigen is SARS-COV-2 S protein (SEQ ID NO:1) or a sequence at least 90% identical thereto. In some embodiments, the subject is a human. In some embodiments, the composition is administered by injection.

### BRIEF DESCRIPTION OF DRAWINGS

FIG. 1 shows the generation of NYVAC-KC-SARS-COV-2 Spike. Parental NYVAC-KC is coumermycin-sensitive (cmr^{S}) and expresses GFP, both in the TK locus. Plasmid DNA encodes the Spike protein surrounded by TK recombination arms. Upon transfection of plasmid and infection with NYVAC-KC, homologous recombination rarely occurs. However, rare recombinant virus expressing Spike can be selected for since recombinant virus is cmr^{R}. Correct recombination is confirmed by loss of green fluorescence.

FIG. 2 shows a schematic of the replication competent NYVAC construct.

Homologous recombination was used to insert a modified gene encoding SARS-COV-2 (Washington strain) Spike into the TK locus of modified NYVAC-KC as depicted in FIG. 1. The spike gene was modified to remove early vaccinia virus transcriptional termination sites, and

NYVAC-KC was modified to express GyrB-PKR from the TK locus.

FIG. 3 shows a gel demonstrating that all seven tested NYVAC-KC-SARS-COV-2-Spike express a protein at the size expected for uncleaved Spike and a second protein at the size expected for cleaved Spike. Lane 1: a negative control, i.e., NYVAC-KC-HIV. Lanes 2-8: NYVAC-KC-SARS-COV-2-Spike candidates 1-7. Lane 9: a positive control, i.e., synthetic SARS-COV-2-Spike.

FIG. 4 shows an agarose gel in which the amplification product (amplified using GyrB-PKR internal primers) that indicates that the spike gene was not inserted into the TK gene of the virus.

FIG. 5 shows an agarose gel in which the amplification product (amplified using GyrB-PKR internal primers) that indicates that the spike gene was inserted into the TK gene of the virus.

FIG. 6 shows a western blot probing for spike protein. The results indicate that all seven tested viruses express uncleaved spike.

FIG. 7 shows the percent of original body weight of mice following vaccination and challenge with a mouse-adapted SARS-COV-2 virus. Mice were vaccinated with a prime and two boosts of the indicated vaccines and body weight was measured for 10 days post-challenge.

FIG. 8 shows clinical scores for the mice tested in FIG. 7.

FIG. 9 shows dilutions of the serum of mice treated with a prime of a plant-derived VLP and two boosts of a replication-competent vaccine vector (i.e., group B).

FIG. 10 shows the SARS-COV-2 neutralizing antibody activity detected in the serum of vaccinated mice at five time points: before prime, one month after prime, one month after the 1st boost, three months after the 1st boost, and two weeks after the 2nd boost.

FIG. 11 shows NYVAC-KC-pfsSpike. NYVAC-KC is a highly attenuated, replication-competent derivative of the Copenhagen strain of vaccinia virus, that has been deleted of 16 open reading frames. A pre-fusion stabilized spike, under control of a synthetic early/late promoter was inserted into the TK locus of NYVAC-KC to generate NYVAC-KC-pfsSpike.

FIG. 12A-12B shows RBD binding antibodies. Serum from animals bled at the indicated times were assayed for the ability to inhibit binding of gold-labeled Washington strain RBD to huACE2. Controls indicated inhibition of binding by a strongly neutralizing positive control, and a weakly neutralizing positive control. FIG. 12A shows sub-cutaneous injection and FIG. 12B shows intranasal administration.

FIG. 13 shows survival after challenge with mouse-adapted SARS-COV-2, SARS2-N501YMA30. Animals either not immunized with NYVAC-KC-pfsSpike or immunized with NYVAC-KC-pfsSpike were challenged with 2×10³ pfu of mouse adapted SARS2-N501YMA30. Animals were monitored for morbidity daily in a blinded manner for up to 10 days (see FIG. 5). Animals with a clinical score of 8 or higher were humanely euthanized.

FIG. 14A-14E shows clinical scores of challenged animals. Animals were monitored for morbidity (weight loss, ruffled fur, hunching, diminished activity, with a range of 0-3 for each parameter, with 0 being no symptoms, 1 indicating mild symptoms, 2 indicating moderate symptoms, and 3 indicating severe symptoms) for up to 10 days after challenge. Animals with an aggregate score of 8 or greater were humanely euthanized. A. Animals not immunized with NYVAC-KC-pfsSpike and not challenged. B. Animals not immunized with NYVAC-KC-pfsSpike and challenged with mouse adapted SARS2-N501YMA30. C. Animals immunized with NYVAC-KC-pfsSpike and challenged with mouse adapted SARS2-N501YMA30. D. Animals immunized by scarification with NYVAC-KC-pfsSpike and challenged with mouse adapted SARS2-N501YMA30. E. Animals immunized intranasally with NYVAC-KC-pfsSpike and challenged with mouse adapted SARS2-N501YMA30.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### DETAILED DESCRIPTION OF THE INVENTION

The present disclosure describes severe acute respiratory syndrome coronavirus 2 (SARS-COV-2) vaccine compositions, as well as methods for making and using the same.

Severe acute respiratory syndrome coronavirus 2 (SARS-COV-2) is the third highly pathogenic human CoV to emerge in the past two decades. The virus causes COVID-19, a severe respiratory disease with an estimated mortality of 2-3% that rapidly spread across China beginning in late 2019 and was declared a world-wide pandemic in early 2020. Like other CoVs, the spike (S) protein is assumed to be the major target for neutralizing antibodies. SARS-COV-2 S protein binds to the receptor, angiotensin-converting enzyme 2 (ACE2), through its receptor binding domain (RBD). The RBDs for other CoVs are immunogenic and a major neutralizing determinant. There are significant concerns that it will become embedded in the viral respiratory disease landscape that will be encountered seasonally. Thus, development of additional safe and effective vaccines against the virus is a significant priority. The long-term goal of this project is to develop a safe, efficacious vaccine(s) against SARS-COV-2. Standard molecular biology, biochemical approaches, vaccination and immunogenicity assessment will be used to generate virus-like-particles (VLPS) and an attenuated vaccinia virus. The goal of this work is to optimally produce VLPs and attenuated vaccinia virus and evaluate immune responses elicited in mice vaccinated with these VLPs or attenuated vaccina viruses.

SARS-COV-2 includes membrane (M), spike (S), envelope (E), and nucleocapsid (N) structural proteins. The M, S, and E proteins provide the structure of the exterior viral envelope.

The S protein is a glycoprotein that mediates receptor binding and fusion during entry into a host cell. The S protein of SARS-COV-2 has the sequence of SEQ ID NO:1. The receptor binding domain (RBD, SEQ ID NO:6) is amino acids 318-510 of SEQ ID NO:1. The annotated DNA sequence encoding SARS-COV2-Spike flanked by TK recombination arms (SEQ ID NO:14) is shown at the end of Example 2.

In some embodiments, the native S protein has been modified to improve its expression from the vaccines described herein. For example, the inventors have generated a modified S protein (SEQ ID NO:15) that includes a mutation in the furin cleavage site (rrar>gsas) and six proline mutations (i.e., F817P, A892P, A899P, A942P, KV986/7>PP) that collectively stabilize the pre-fusion S protein.

In some embodiments, the DNA sequence encoding the S protein is codon-optimized for expression in a particular species. For example, in some embodiments, the S protein is encoded by SEQ ID NO: 16. The M protein of SARS-COV-2 has the sequence of SEQ ID NO:7. The E protein of SARS-COV-2 has the sequence of SEQ ID NO:8. The N protein is an internal structural component that encapsulates the SARS-COV-2 viral genome. The N protein of SARS-COV-2 has the sequence of SEQ ID NO:9.

As used herein, “SARS-COV-2 antigen” refers to a SARS-COV-2 protein, a sequence at least 90% identical thereto, a fragment thereof, or combinations thereof that may be used to elicit an immune response in a subject. The SARS-COV-2 antigen may be the SARS-COV-2 S protein, the SARS-COV-2 M protein, the SARS-COV-2 E protein, the SARS-COV-2 N protein, the SARS-CoV-2 S protein RBD, a protein with a sequence at least 90%, 95%, 98%, or 99% sequence identity thereto, or combinations thereof. The SARS-COV-2 virus has continued to evolve over the past two years and many mutations in the Spike protein identified. Several of these mutations in SARS-COV-2 Spike or RBD may be included in the vaccines described herein and include SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, or SEQ ID NO: 37. Each of these mutant Spike sequences can be used in combination with the modifications described above to allow for improved expression from the vaccines described here.

Notably SEQ ID NO: 22-41 contain individual mutations identified in the S protein and these individual mutations may be combined to form novel S proteins that may be used to generate the vaccines and VLPs described herein. For example, the delta variant of the virus contains the mutations provided in SEQ ID NO: 31 and 33 in combination. Thus, these described combinations as well as new combinations are also provided herein. It is likely that new combinations of mutations in the S protein will continue to arise in the circulating virus population and the vaccines and VLPs described herein will need to take account of the circulating virus in order to maintain immunogenicity.

As used herein, the phrases “% sequence identity,” “percent identity,” or “% identity” are used interchangeably and refer to the percentage of residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. A suite of commonly used and freely available sequence comparison algorithms is provided by the National Center for Biotechnology Information (NCBI) Basic Local Alignment Search Tool (BLAST® alignment tool), which is available from several sources, including the NCBI, Bethesda, Md., at its website. The BLAST® alignment tool software suite includes various sequence analysis programs including “blastp,” that is used to align a known amino acid sequence with other amino acids sequences from a variety of databases.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Polynucleotides encoding any of the SARS-COV-2 antigens described herein are provided. As used herein, the terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of natural or synthetic origin (which may be single-stranded or double-stranded and may represent the sense or the antisense strand). The polynucleotides may be cDNA or genomic DNA.

Polynucleotides homologous to the polynucleotides described herein are also provided. Those of skill in the art understand the degeneracy of the genetic code and that a variety of polynucleotides can encode the same polypeptide. In some embodiments, the polynucleotides (i.e., polynucleotides encoding the SARS-COV-2 antigens described herein) may be codon-optimized for expression in a particular cell including, without limitation, a plant cell, mammalian cell, insect cell, bacterial cell, or fungal cell. While particular polynucleotide sequences are disclosed herein, any polynucleotide sequences may be used which encodes a desired form of the polypeptides described herein. Thus non-naturally occurring sequences may be used. These may be desirable, for example, to enhance expression in heterologous expression systems of polypeptides or proteins. Computer programs for generating degenerate coding sequences are available and can be used for this purpose. Pencil, paper, the genetic code, and a human hand can also be used to generate degenerate coding sequences.

In another aspect of the present invention, constructs are provided. As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies.

The constructs provided herein may be prepared by methods available to those of skill in the art. Notably each of the constructs claimed are recombinant molecules and as such do not occur in nature. Generally, the nomenclature used herein and the laboratory procedures utilized in the present invention include molecular, biochemical, and recombinant DNA techniques that are well known and commonly employed in the art. Standard techniques available to those skilled in the art may be used for cloning, DNA and RNA isolation, amplification and purification. Such techniques are thoroughly explained in the literature.

The constructs provided herein may include a promoter operably linked to any one of the polynucleotides described herein. As used herein, a polynucleotide is “operably connected” or “operably linked” when it is placed into a functional relationship with a second polynucleotide sequence.

As used herein, the terms “heterologous promoter,” “promoter,” “promoter region,” or “promoter sequence” refer generally to transcriptional regulatory regions of a gene, which may be found at the 5′ or 3′ side of a polynucleotides described herein, or within the coding region of said polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

Heterologous promoters useful in the practice of the present invention include, but are not limited to, constitutive, inducible, temporally-regulated, developmentally regulated, chemically regulated, tissue-preferred and tissue-specific promoters. The heterologous promoter may be a plant, animal, bacterial, fungal, or synthetic promoter. Suitable promoters are known and described in the art. Suitable promoters for expression in plants include, without limitation, the 35S promoter of the cauliflower mosaic virus, ubiquitin, tCUP cryptic constitutive promoter, the Rsyn7 promoter, pathogen-inducible promoters, the maize In2-2 promoter, the tobacco PR-1a promoter, glucocorticoid-inducible promoters, estrogen-inducible promoters and tetracycline-inducible and tetracycline-repressible promoters. Other promoters include the T3, T7 and SP6 promoter sequences, which are often used for in vitro transcription of RNA. In mammalian cells, typical promoters include, without limitation, promoters for Rous sarcoma virus (RSV), human immunodeficiency virus (HIV-1), cytomegalovirus (CMV), SV40 virus, and the like as well as the translational elongation factor EF-1α promoter or ubiquitin promoter.

In some embodiments, the promoter is viral synthetic late promoter (SLP). In some embodiments, the SLP has the sequence of SEQ ID NO:5. Those of skill in the art are familiar with a wide variety of additional promoters for use in various cell types.

The constructs provided herein may include a translation enhancing element (TEE) operably linked to any one of the polynucleotides described herein.

As used herein “translation enhancing elements (TEE),” refers to polynucleotide sequences that mediate cap-independent translation initiation. A TEE polynucleotide refers to both the RNA polynucleotide being translated and the DNA polynucleotide encoding said RNA polynucleotide. Identification of TEEs is described in US Publication No. 20130230884 and described by Wellensiek et al. (“Genome-wide profiling of cap-independent translation enhancing elements in the human genome,” Nat Methods, 2013, 10(8):747-750). Suitable TEEs are also described in US Publication No. 20140255990 and Wellensiek et al. (“A leader sequence capable of enhancing RNA expression and protein synthesis in mammalian cells,” Protein Sci., 2013, 22(10): 1392-1398). In some embodiments, the TEE includes the sequence of SEQ ID NO:4. In some embodiments, the TEE includes the sequence of SEQ ID NO: 10. In some embodiments, the TEE includes the sequence of SEQ ID NO:11. In some embodiments, the TEE includes the sequence of SEQ ID NO:12. In some embodiments, a polynucleotide sequence may act as both a promoter and a TEE.

Vectors including any of the constructs or polynucleotides described herein are provided. The term “vector” is intended to refer to a polynucleotide capable of transporting another polynucleotide to which it has been linked. In some embodiments, the vector may be a “plasmid,” which refers to a circular double-stranded DNA loop into which additional DNA segments may be ligated. Certain vectors are capable of autonomous replication in a host cell into which they are introduced (e.g., bacterial vectors having a bacterial origin of replication and episomal mammalian vectors). Other vectors can be integrated into the genome of a host cell upon introduction into the host cell, and thereby are replicated along with the host genome, such as some viral vectors or transposons. Viral genomes are also included as vectors, including vectors based on viral genomes. Vectors may carry genetic elements, such as those that confer resistance to certain drugs or chemicals.

In some aspects, the vector is a vaccinia virus expression vector based on the vaccinia virus genome. Vaccinia virus (VACV or VV) is a large, complex, enveloped virus belonging to the poxvirus family. It has a linear, double-stranded DNA genome of approximately 190 kb in length, which encodes around 250 genes. The genome is surrounded by a lipoprotein core membrane. The poxviruses are the largest known DNA viruses and are distinguished from other viruses by their ability to replicate entirely in the cytoplasm of the host cell, outside of the nucleus. VV can accept as much as 25 kb of foreign DNA, making it useful for expressing large genes. Foreign genes are integrated stably into the viral genome, resulting in efficient gene expression. Other viral expression vectors for use in the present invention include, but are not limited to, certain highly attenuated, host-restricted, non- or poorly replicating poxvirus strains have been developed for use as substrates in recombinant vaccine development. These strains include the Orthopoxviruses, Modified Vaccinia Ankara (MVA) and NYVAC (derived from the Copenhagen vaccinia strain), and the Avipoxviruses, ALVAC and TROVAC (derived from canarypox and fowlpox viruses, respectively). In some embodiments, the viral expression vectors described herein may be modified to have one or more desirable properties.

In some embodiments, the viral expression vector is a NYVAC vector that has been modified to be replication-competent with improved T cell and antibody responses to the delivered antigen. As used herein “NYVAC-KC” refers to a NYVAC vector modified to include a polynucleotide encoding the C7L polypeptide (SEQ ID NO:2) adjacent to a polynucleotide encoding the KIL polypeptide (SEQ ID NO:3). Both C7L and K1L have been shown to be involved in defining the replication competence of the virus. The NYVAC-KC vector is described in further detail in U.S. Pat. No. 9,670,506, which is incorporated herein by reference in its entirety.

In some embodiments, vectors described herein include an internal ribosomal entry site (IRES). IRES is an RNA element that recruits eukaryotic ribosome and allows for translation initiation in a cap-independent manner, often located in the 5′UTR, but can also occur elsewhere in the mRNA. In some embodiments, vectors described herein include at least two IRES. In some embodiments, vectors described herein include a self-cleaving protein element. Self-cleaving peptides induce ribosomal skipping during translation, causing the ribosome to fail at making a peptide bond causing an apparent cleave. Self-cleaving peptides include the 2A class of peptides. In some embodiments, vectors described herein include at least two self-cleaving protein elements.

In some aspects, provided herein are virus-like particles (VLPs) or recombinant immune complexes incorporating the SARS-COV-2 antigens described herein.

As used herein, “virus-like particles (VLPs)” refers to particles that include one or more viral proteins and mimics the structure of the native virus but lack the viral genome. In some embodiments, the VLP includes at least the S protein. In some embodiments, the VLP includes at least the M and E proteins. In some embodiments, the VLP includes at least the M, E, and S proteins. In some embodiments, the VLP includes the M, E, S, and N proteins.

Vaccine compositions including the SARS-COV-2 antigens or VLPs described herein are also provided. As used herein “vaccine” refers to a composition that includes an antigen. Vaccine may also include a biological preparation that improves immunity or the immune response to a particular disease. A vaccine may typically contain an agent, referred to as an antigen, that resembles or is a part of a disease-causing microorganism, in this case SARS-COV-2, and the agent may often be made from weakened or killed forms of the microbe, its toxins or one of its surface proteins. The antigen may stimulate the body's immune system to recognize the agent as foreign, destroy it, and “remember” it, so that the immune system can more easily recognize and destroy any of these microorganisms that it later encounters.

Vaccines may be prophylactic, e.g., to prevent or ameliorate the effects of a future infection by any natural or “wild” pathogen, or therapeutic, e.g., to treat the disease. Administration of the vaccine to a subject results in an immune response, generally against one or more specific diseases. The amount of a vaccine that is therapeutically effective may vary depending on the particular virus used, or the condition of the patient, and may be determined by a physician. The vaccine may be introduced directly into the subject by the intramuscular, intravenous, subcutaneous, oral, oronasal, or intranasal routes of administration.

The vaccine compositions described herein also include a suitable carrier or vehicle for delivery. As used herein, the term “carrier” refers to a pharmaceutically acceptable solid or liquid filler, diluent or encapsulating material. A water-containing liquid carrier can contain pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories, may be found in the U.S. Pharmacopeia National Formulary, 1857-1859, (1990).

Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator.

Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

In another embodiment, the present formulation may also comprise other suitable agents such as a stabilizing delivery vehicle, carrier, support or complex-forming species. The coordinate administration methods and combinatorial formulations of the instant invention may optionally incorporate effective carriers, processing agents, or delivery vehicles, to provide improved formulations for delivery of the SARS-COV-2 antigens or VLPs described herein.

The vaccine formulation may additionally include a biologically acceptable buffer to maintain a pH close to neutral (7.0-7.3). Such buffers preferably used are typically phosphates, carboxylates, and bicarbonates. More preferred buffering agents are sodium phosphate, potassium phosphate, sodium citrate, calcium lactate, sodium succinate, sodium glutamate, sodium bicarbonate, and potassium bicarbonate. The buffer may comprise about 0.0001-5% (w/v) of the vaccine formulation, more preferably about 0.001-1% (w/v). Other excipients, if desired, may be included as part of the final vaccine formulation.

In some embodiments, the present formulation may also comprise an adjuvant. An adjuvant is a substance or combination of substances that is used to increase the efficacy or potency of the formulation or modulates the immune response to a vaccine. An adjuvant may accelerate, prolong or enhance antigen-specific immune responses when used in combination with an antigen.

An adjuvant may be an inorganic compound such as potassium alum, aluminum hydroxide, aluminum phosphate or calcium phosphate hydroxide. An adjuvant may also be an oil such as paraffin oil or propolis, a bacterial product such as killed Bordetella pertussis or Mycobacterium bovis or their toxoids, monophosphoryl lipid A or detoxified Salmonella spp. lipopolysaccharide. An adjuvant may also be derived from plants, such as saponins from Quillaja (QS-21), soybean or Polygala senega. Cytokines such as IL-1, IL-2 or IL-12 may also act as adjuvants. Adjuvants may also include Freund's complete or incomplete adjuvant or squalene including AS03 or MF59.

The remainder of the vaccine formulation may be an acceptable diluent, to 100%, including water. The vaccine formulation may also be formulated as part of a water-in-oil, or oil-in-water emulsion.

The vaccine formulation may be separated into vials or other suitable containers. The vaccine formulation herein described may then be packaged in individual or multi-dose ampoules or be subsequently lyophilized (freeze-dried) before packaging in individual or multi-dose ampoules. The vaccine formulation herein contemplated also includes the lyophilized version. The lyophilized vaccine formulation may be stored for extended periods of time without loss of viability at ambient temperatures. The lyophilized vaccine may be reconstituted by the end user and administered to a patient.

The term “lyophilization” or “lyophilized,” as used herein, refers to freezing of a material at low temperature followed by dehydration by sublimation, usually under a high vacuum. Lyophilization is also known as freeze drying. Many techniques of freezing are known in the art of lyophilization such as tray-freezing, shelf-freezing, spray-freezing, shell-freezing and liquid nitrogen immersion. Each technique will result in a different rate of freezing. Shell-freezing may be automated or manual. For example, flasks can be automatically rotated by motor driven rollers in a refrigerated bath containing alcohol, acetone, liquid nitrogen, or any other appropriate fluid. A thin coating of product is evenly frozen around the inside “shell” of a flask, permitting a greater volume of material to be safely processed during each freeze drying run. Tray-freezing may be performed by, for example, placing the samples in lyophilizer, equilibrating 1 hr at a shelf temperature of 0° C., then cooling the shelves at 0.5° C./min to −40° C. Spray-freezing, for example, may be performed by spray-freezing into liquid, dropping by ˜20 μl droplets into liquid N₂, spray-freezing into vapor over liquid, or by other techniques known in the art.

Methods of inducing an immune response in a subject are also provided. A vaccine composition as described herein and including a SARS-COV-2 antigen or VLP as described herein is administered to a subject to induce an immune response. Following administration, the immune response of the subject may be tested using methods known in the art.

To vaccinate a subject, a therapeutically effective amount of a vaccine composition described herein is administered to the subject. The therapeutically effective amount of vaccine may typically be one or more doses, preferably in the range of about 0.01-10 mL, most preferably 0.1-1 mL, containing 1-500 micrograms, most preferably 1-100 micrograms of vaccine formulation/dose. The therapeutically effective amount may also depend on the vaccination species. For example, for smaller animals such as mice, a preferred dosage may be about 0.01-1 mL of a 1-50 microgram solution of antigen. For a human patient, a preferred dosage may be about 0.1-1 mL of a 1-50 microgram solution of antigen. The therapeutically effective amount may also depend on other conditions including characteristics of the patient (age, body weight, gender, health condition, etc.), and others.

The term “administration,” as used herein, refers to the introduction of a substance, such as a vaccine, into a subject's body. The administration, e.g., parenteral administration, may include subcutaneous administration, intramuscular administration, transcutaneous administration, intradermal administration, intraperitoneal administration, intraocular administration, intranasal administration, oral administration and intravenous administration.

The vaccine or the composition according to the invention may be administered to an individual according to methods known in the art. Such methods comprise application e.g. parenterally, such as through all routes of injection into or through the skin: e.g. intramuscular, intravenous, intraperitoneal, intradermal, mucosal, submucosal, or subcutaneous. Also, the vaccine may be applied by topical application as a drop, spray, gel or ointment to the mucosal epithelium of the eye, nose, mouth, anus, or vagina, or onto the epidermis of the outer skin at any part of the body.

Other possible routes of application are by spray, aerosol, or powder application through inhalation via the respiratory tract. In this last case, the particle size that is used will determine how deep the particles will penetrate into the respiratory tract.

Alternatively, application may be via the alimentary route, by combining with the food, feed or drinking water e.g. as a powder, a liquid, or tablet, or by administration directly into the mouth as a: liquid, a gel, a tablet, or a capsule, or to the anus as a suppository.

The present disclosure is generally applied to mammals, including but not limited to humans, cows, horses, sheep, pigs, goats, rabbits, dogs, cats, bats, mice and rats. In some embodiments, the present disclosure can be applied to birds. In certain embodiments, non-human mammals, such as mice and rats, may also be used for the purpose of demonstration. One may use the present invention for veterinary purpose. For example, one may wish to treat commercially important farm animals, such as cows, horses, pigs, rabbits, goats, sheep, and birds, such as chickens. One may also wish to treat companion animals, such as cats and dogs.

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, the terms “approximately” or “about” in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

The present invention has been described in terms of one or more preferred embodiments, and it should be appreciated that many equivalents, alternatives, variations, and modifications, aside from those expressly stated, are possible and within the scope of the invention.

### EXAMPLES

The following example describes methods for making and using attenuated, replication-competent, recombinant poxvirus vaccine compositions expressing SARS-COV-2 proteins, VLPs, and immune complexes.

### Example 1—Vaccine Design

All CoVs have at least three envelope structural proteins: the membrane (M), spike (S), and envelope (E) protein. The nucleocapsid (N) protein is an internal structural component that encapsulates the viral genome. The S protein is a glycoprotein that mediates receptor binding and fusion during entry into host cells. Angiotensin-converting enzyme 2 (ACE2) is the receptor for SARS-COV-2 and also SARS-COV [26-28]. S protein is the major target for neutralizing antibodies [29-32]. Many avian and mammalian coronavirus S proteins are cleaved into S1 and S2 [33]. The receptor binding domain (RBD) on S proteins has been mapped for a number of CoVs, including SARS-COV S [34, 35]. Sequence comparison of SARS-COV and SARS-COV-2 S proteins indicates approximately 76% identity for the full length S proteins, ˜73% identify for the RBD, but only ˜50% identify for the receptor binding motif core structure in the RBD [27].

This suggests that while both viruses use ACE2 for entry when infecting cells, receptor interactions may be structurally different, which could require specific targeting for effective vaccine development against SARS-COV-2.

The rationale behind this project is that a live, highly-attenuated, replication-competent vaccinia virus vectored vaccine, expressing SARS-Coronavirus-2 antigens, will give the protection afforded by a live, attenuated vaccine, without the adverse reactions and safety issues associated with many live, attenuated vaccines. While a vaccine based on a live, attenuated coronavirus might be attractive, use of such a coronavirus brings up concerns of reversion and security [76]. As an alternative we are proposing to use a live, replication-competent, highly-attenuated vaccinia virus vector, NYVAC-KC [22], that we have shown to be highly immunogenic [21], and compare it to a replication-deficient MVA based vaccine vector, which has been used to generate SARS and MERS candidate vaccines [77] [78]. We have shown that NYVAC-KC gives superior T cell and antibody responses to HIV antigens, compared to its replication deficient parent, NYVAC [21]. Thus, we expect NYVAC-KC to give superior immune responses compared to the replication-deficient MVA. We will increase immunogenicity of these vectors by including a novel poxvirus transcriptional/translational enhancer (TEE) that we have identified [79], which can increase protein expression 10-20-fold higher than a corresponding optimized poxvirus late promoter. The goal is to generate a vector that can be used with multiple SARS-Coronavirus-2 antigens to mimic immunization with a live attenuated SARS-Coronavirus-2 vector, without the reversion and security concerns of a live attenuated SARS-Coronavirus-2 vaccine.

NYVAC-KC has been engineered to be able to rapidly insert genes into the TK locus [80]. We have inserted a negative selectable marker, encoding sensitivity to the antibiotic coumermycin, along with GFP, into the TK locus of NYVAC-KC (NYVAC-KC-TK:GFP/cmrS). DNA constructs encoding SARS-coronavirus-2 antigens (with late vaccinia virus transcriptional termination sites removed) expressed from a vaccinia virus optimized early/late promoter, surrounded by TK flanking arms have been designed and ordered. We will perform in vivo recombination [22], transfecting with linear DNA and infecting with NYVAC-KC-TK:GFP/cmrS. Virus that has replaced the GFP/cmrS cassette in the TK locus with the SARS-Coronavirus-2 cassette will be colorless and cmrR. Candidate GFP-/cmrR plaques will be picked, and correct insertion will be confirmed by PCR. Plaques will be amplified to P2 in our GLP certified vaccine room, to generate a pre-master-seed stock. Within one month of receiving DNA we can generate a 1010 pfu GLP pre-master-seed stock. Pre-master-seed stocks will be assayed for antigen expression (% antigen positive plaques at P2), sterility, mycoplasma contamination and stability of insert (% antigen positive plaques at P2 vs P9), prior to release for manufacture at GMP. We have successfully generated 5 GLP NYVAC-KC pre-master-seed stocks that have been released for manufacture at GMP.

We will similarly engineer MVA to be cmrS and express GFP from deletion 11. Once we have generated this virus, we will insert SARS-Coronavirus-2 antigens, as described for NYVAC-KC.

We will initially express SARS-Coronavirus-2 Spike (S) from NYVAC-KC and MVA. SARS and MERS S has been shown to induce neutralizing antibodies that are protective in animal model challenge studies [82]. However, particulate antigens are often more immunogenic than soluble or membrane bound antigens. Thus, we will also express the SARS-Coronavirus-2 proteins (e.g., the membrane (M), envelope (E), and nucleocapsid (N) proteins) that are found to be necessary to generate bona fide SARS-Coronavirus-2 VLPs

To express multiple proteins from NYVAC-KC, which will be needed for VLPs, we use tandem promoters driving transcription in opposite directions. We have used this design to express HIV env in one direction, and an HIV gag-pol-nef fusion protein in the opposite direction. Thus, we have successfully expressed 7 KB of transgenes from NYVAC-KC. We can enhance the number of independent proteins expressed from this construct either using optimized IRESs [84], or self-cleaving protein elements [85], [86].

All constructs will be analyzed for transgene expression by Western blotting, after single-cycle and multi-cycle infections. Analysis of multi-cycle infections will provide an estimate of the increase in gene expression we might expect from a replication-competent vector. For VLPs and immune complex antigens, supernatants and cytoplasmic extracts will be collected, and particulate matter will be purified by ultracentrifugation through a sucrose pad, followed by Western blot analysis and immune-microscopy.

### Example 2—Vaccine Generation

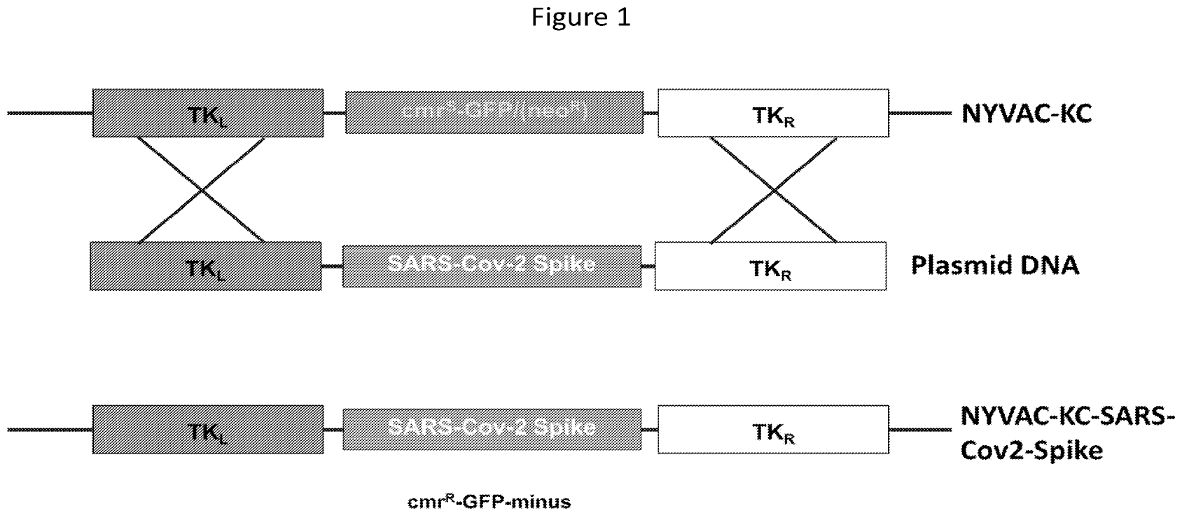

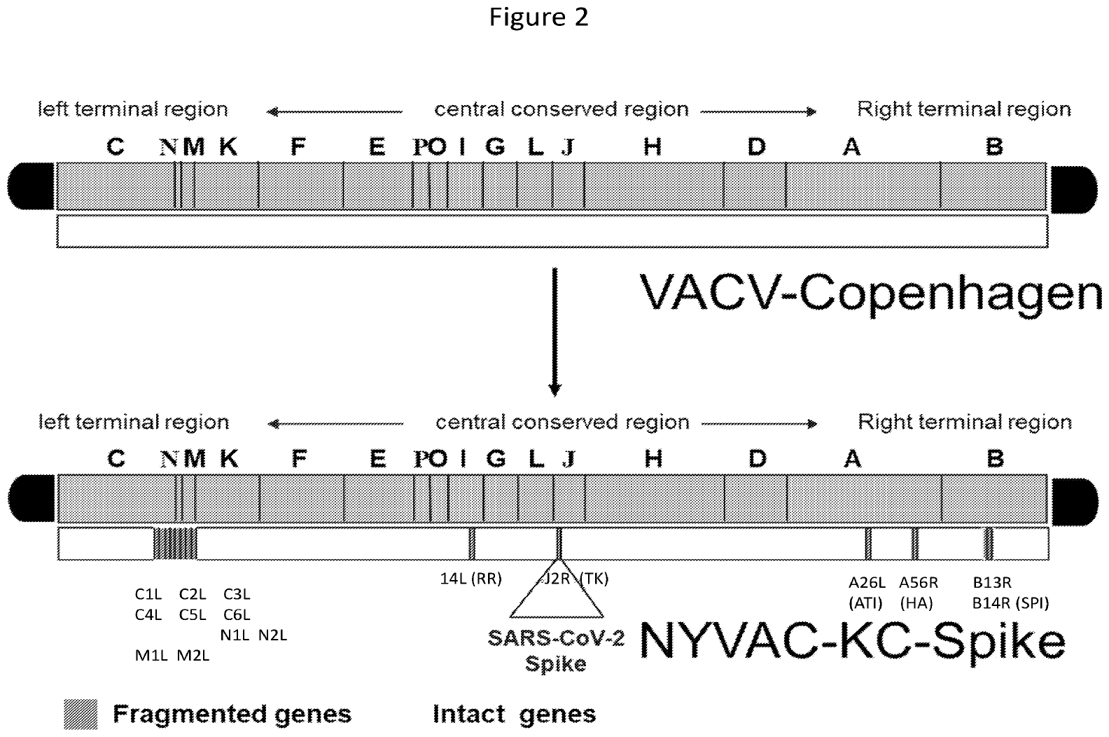

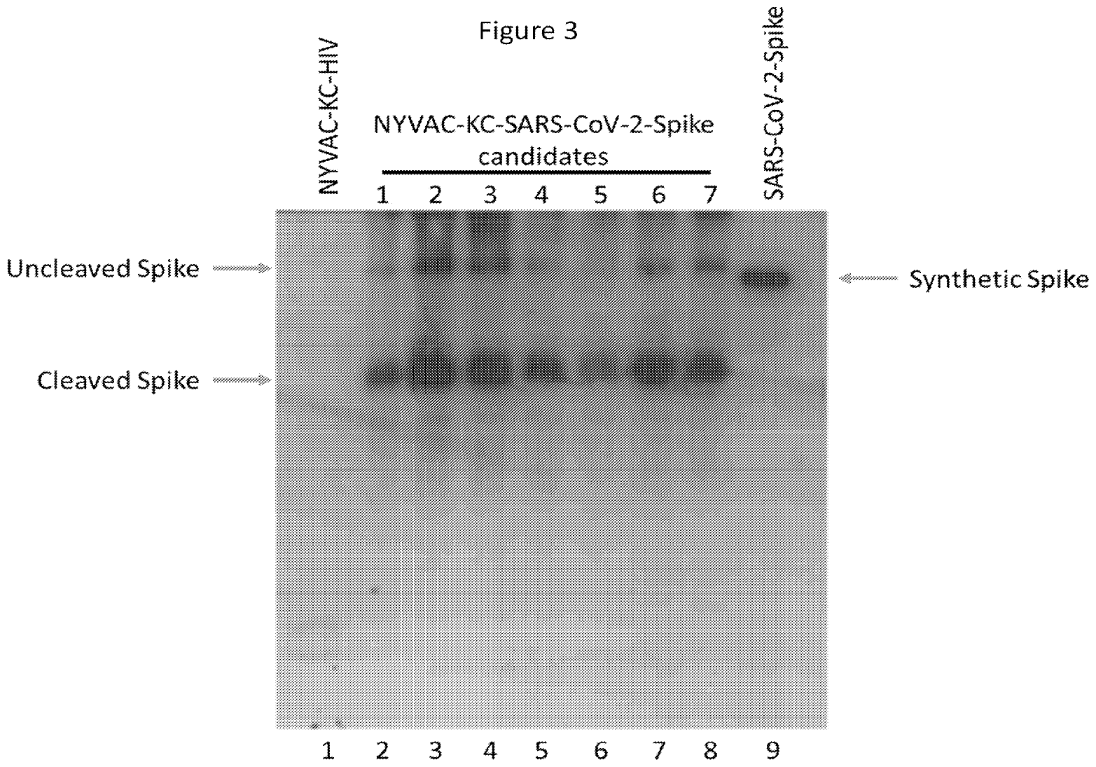

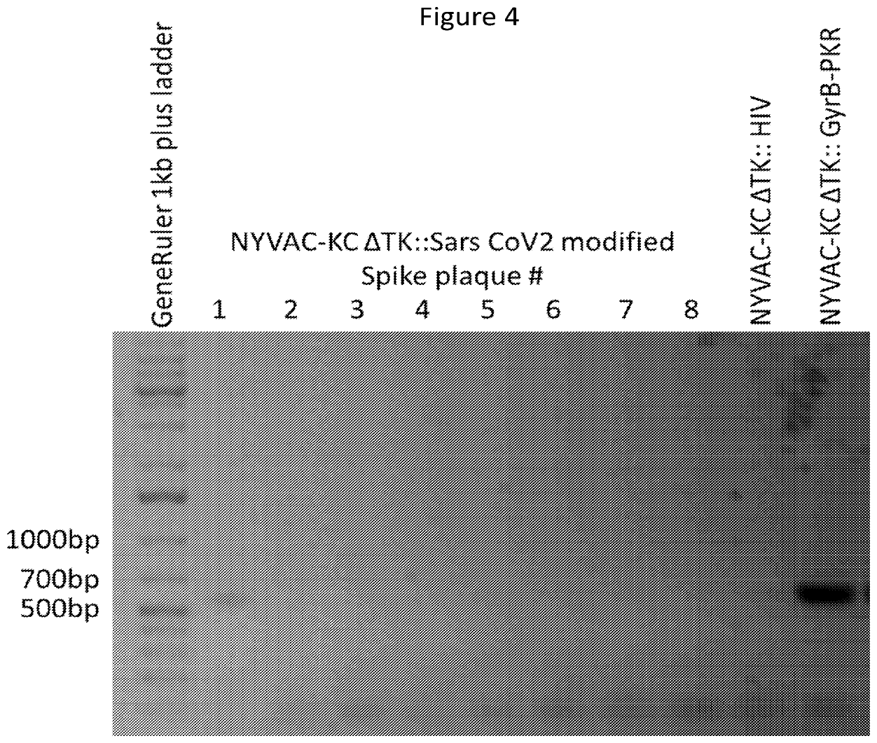

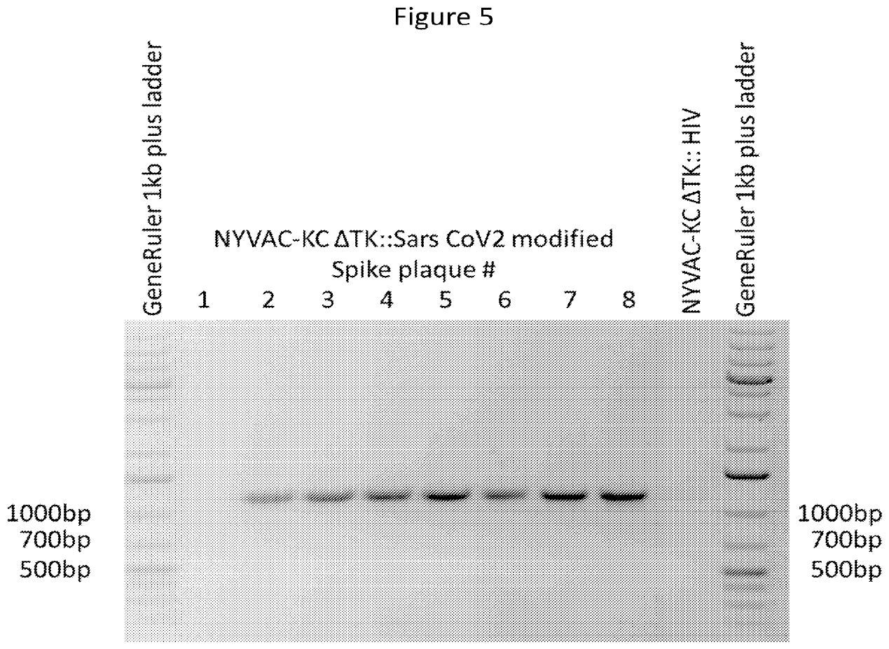

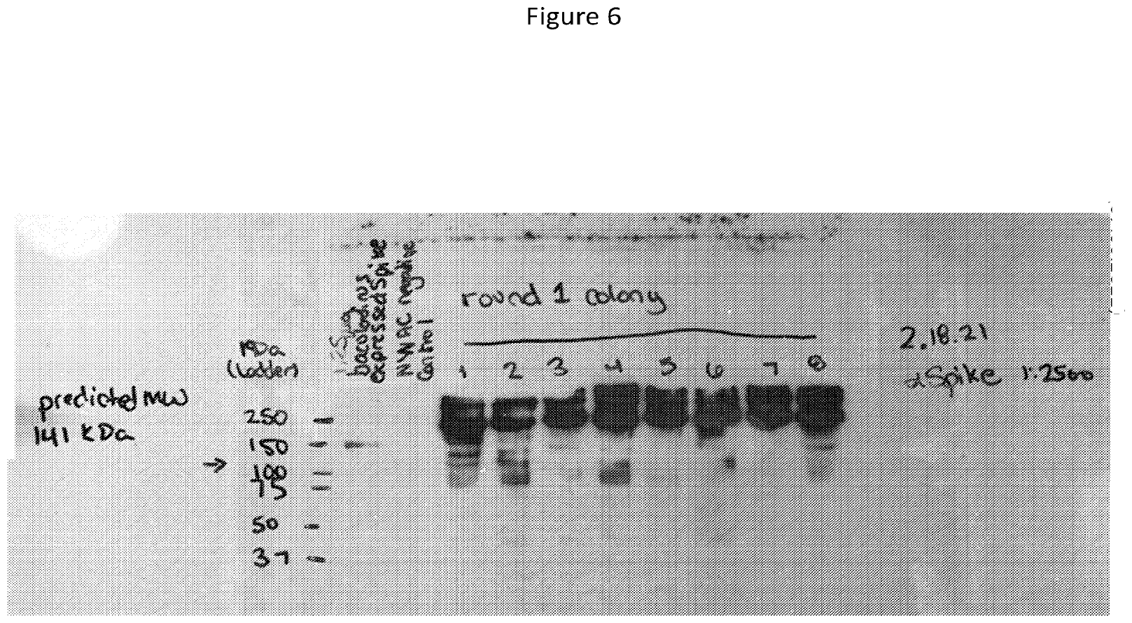

We have used our rapid vaccinia virus recombinant generation system to generate 7 candidate clones of NYVAC-KC-SARS-COV-2-Spike, as depicted in FIG. 1. The parental virus, NYVAC-KC, has a coumermycin-sensitivity (cmrS)/GFP cassette in the TK locus of the virus. The cassette has arms that are homologous to the sequence flanking the TK deletion in NYVAC-KC, to allow for in vivo recombination with the viral genome, and an E/L promoter. We have obtained DNA encoding SARS-COV2-Spike flanked by TK recombination arms. Rare replacement of the cmrS/GFP cassette in NYVAC-KC with the SARS-COV-2 spike encoding DNA will lead to a selectable cmrR/non-GFP recombinant virus. Seven cmrR/non-GFP plaques were picked from the original homologous recombination reactions. Two, plaques 1 and 3, had no green plaques on subsequent plaque purification, and no cmrS/GFP cassette could be detected by PCR. The remaining 5 plaques appeared to still contain some parental virus, which is in the process of being removed by plaque purification. All 7 candidates express a protein at the size expected for uncleaved Spike and a second protein at the size expected for cleaved Spike (FIG. 3). Notably, synthetic Spike was generated in insect cells and is likely poorly glycosylated, accounting for the slight difference in electrophoretic mobility between synthetic Spike and uncleaved Spike expressed from NYVAC-KC. The results are supported by western blot analysis (FIG. 6). Clones 1 and 3 were amplified to generate enough virus to vaccinate animals.

All stocks of NYVAC-KC-SARS-COV-2-Spike were generated in a good laboratory practice (GLP) vaccine room. We started to make this virus before all GLP materials had arrived. Current stocks will be used for animal experiments. While animal experiments are in progress with existing virus, new, GLP stocks of NYVAC-KC-SARS-COV-2-Spike will be generated.

### Example 3—Challenge Experiment

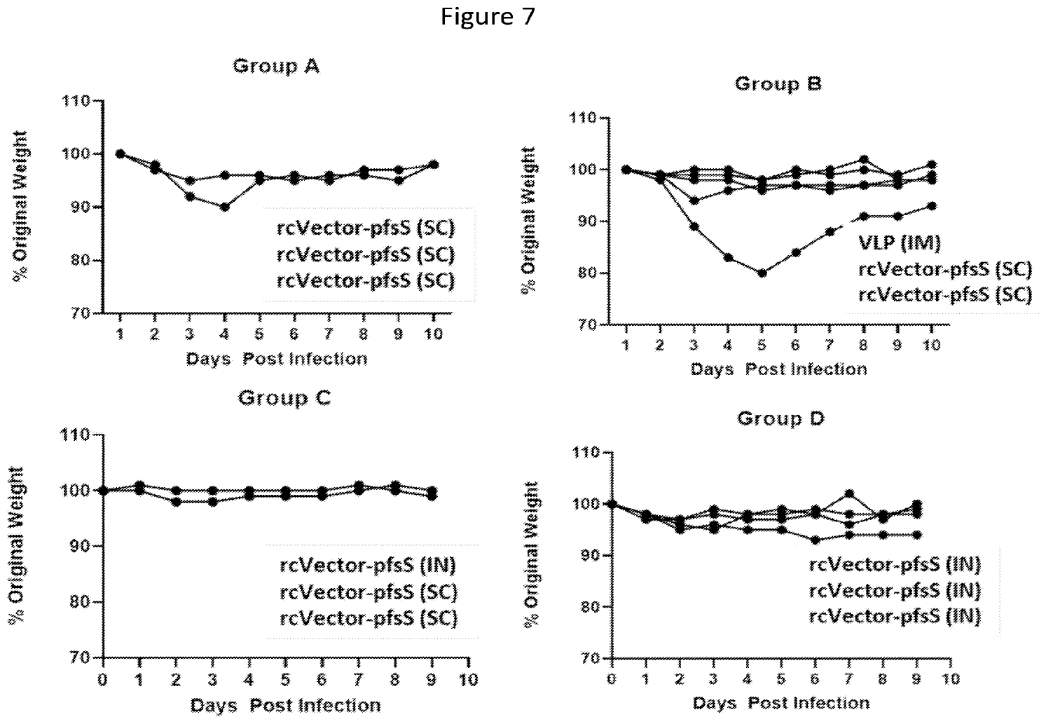

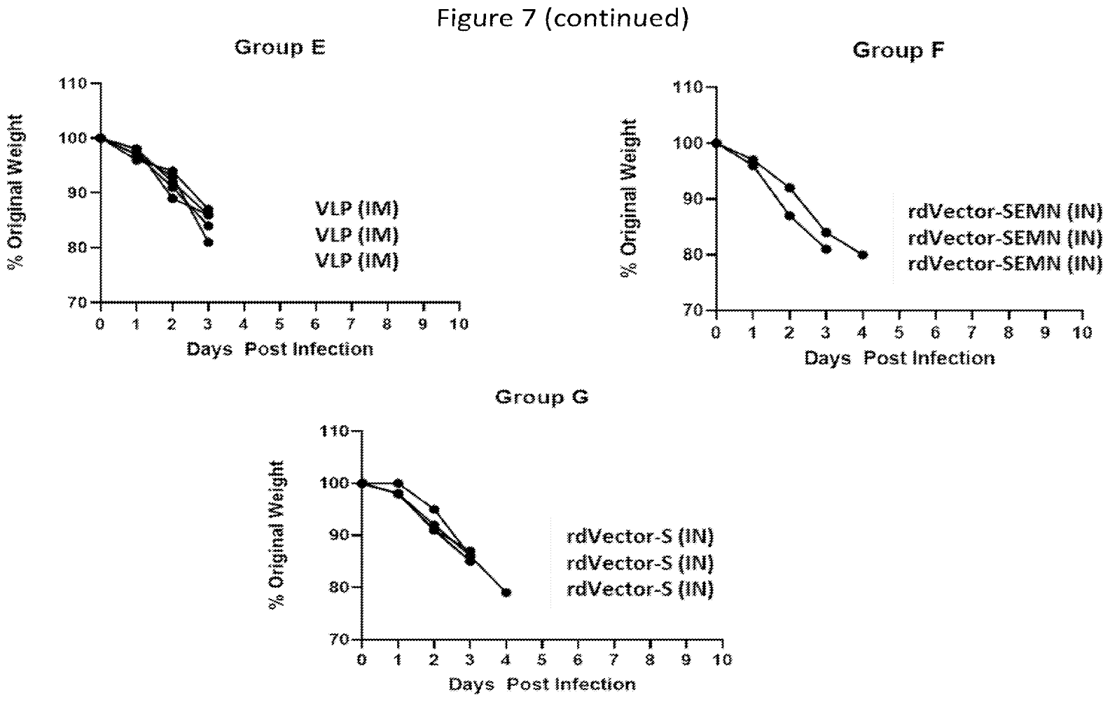

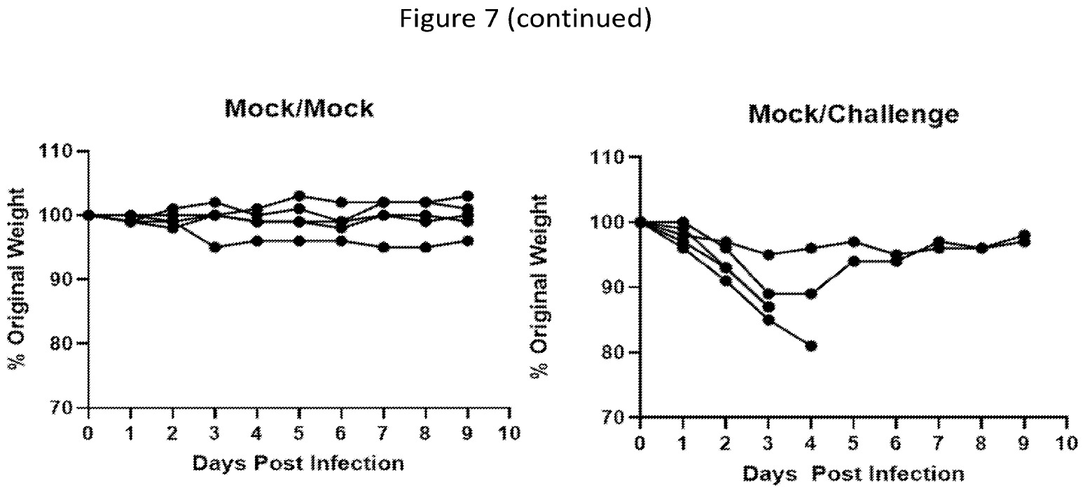

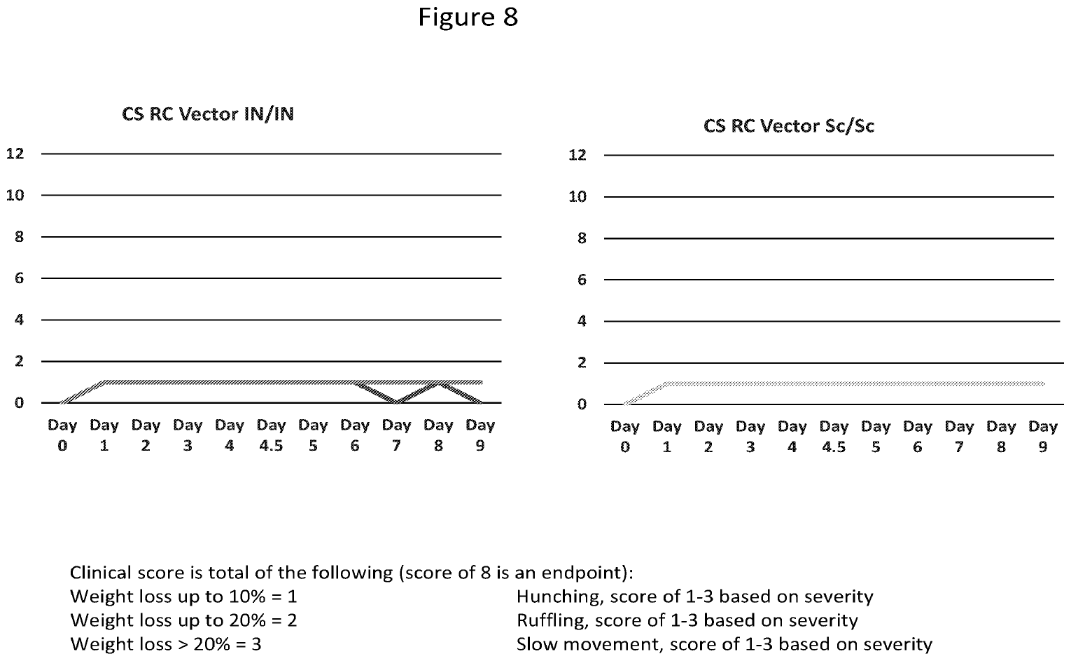

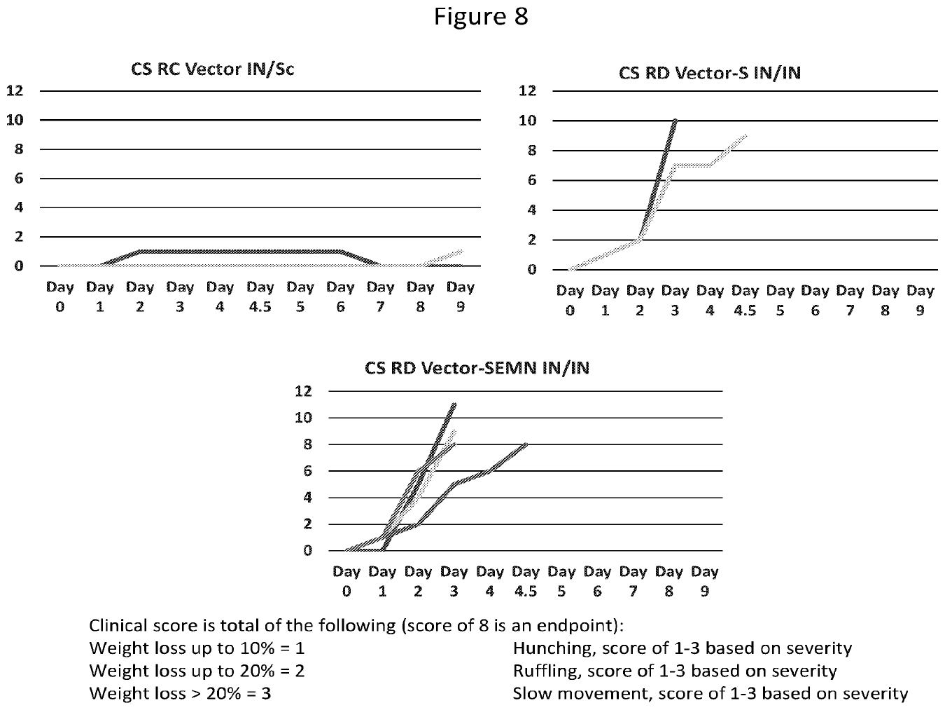

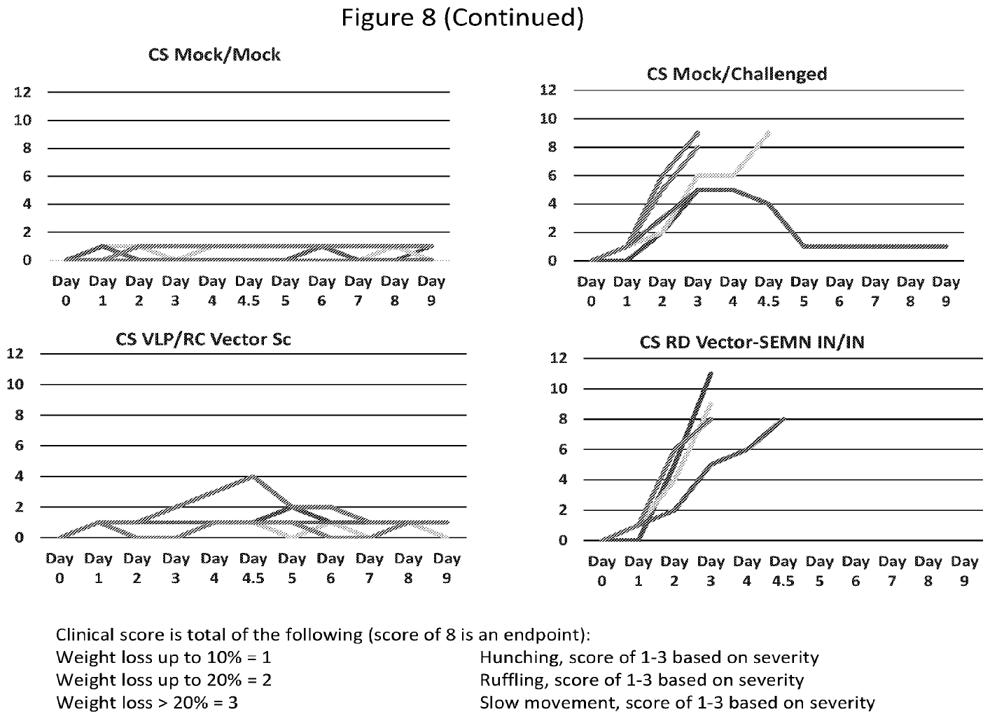

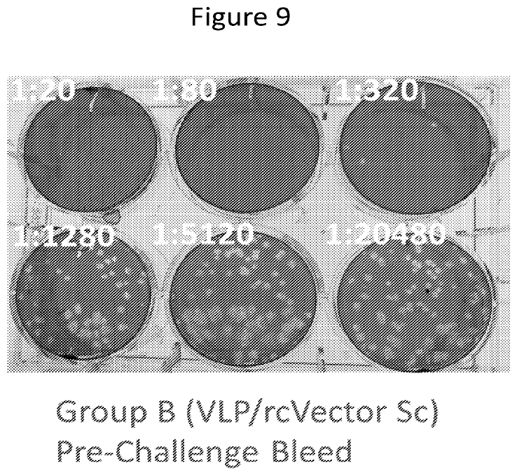

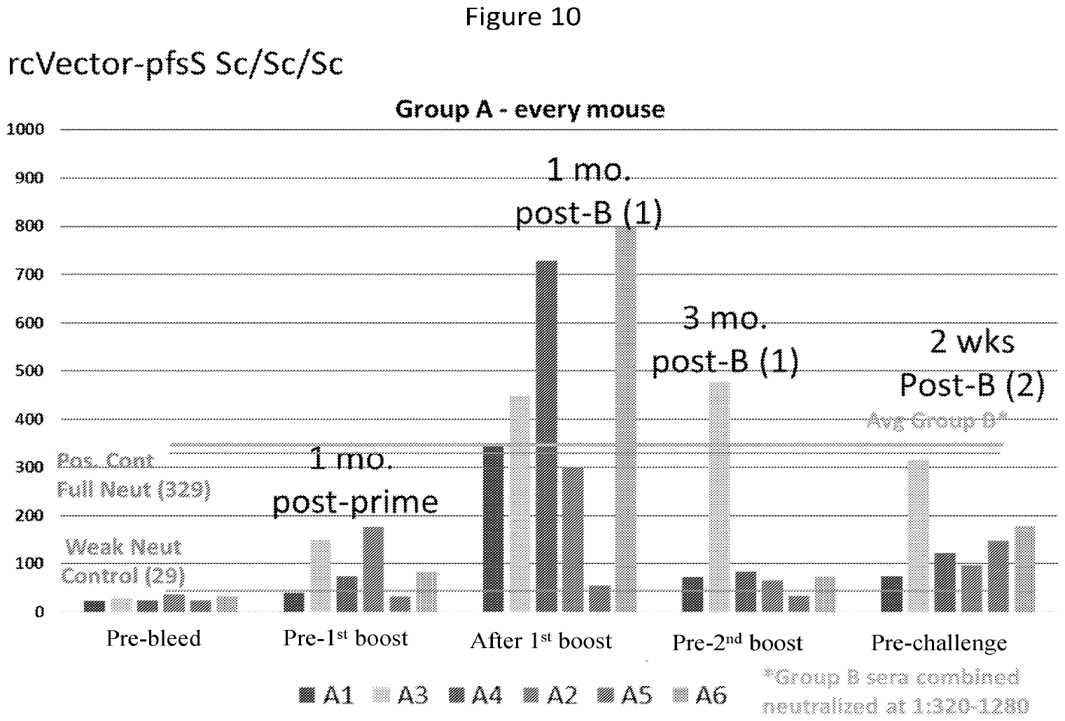

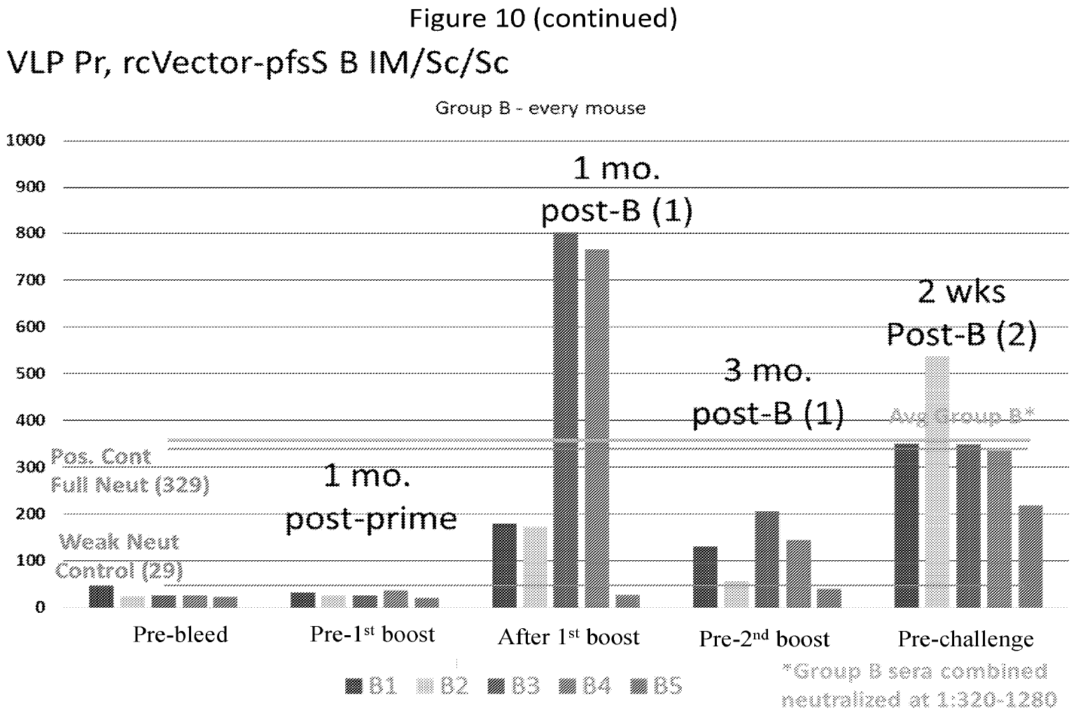

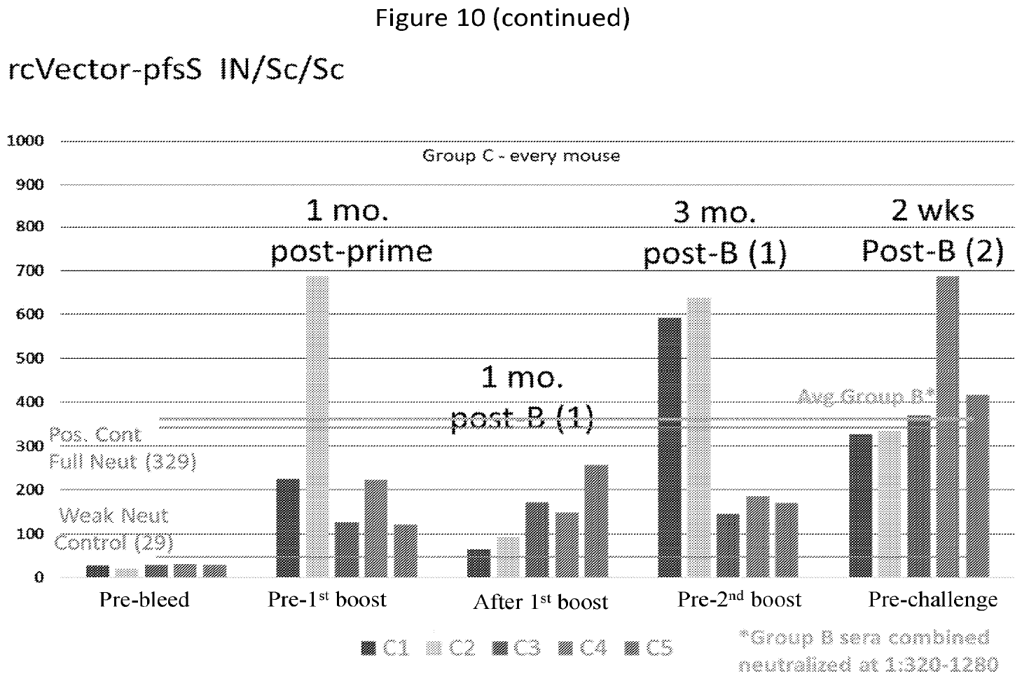

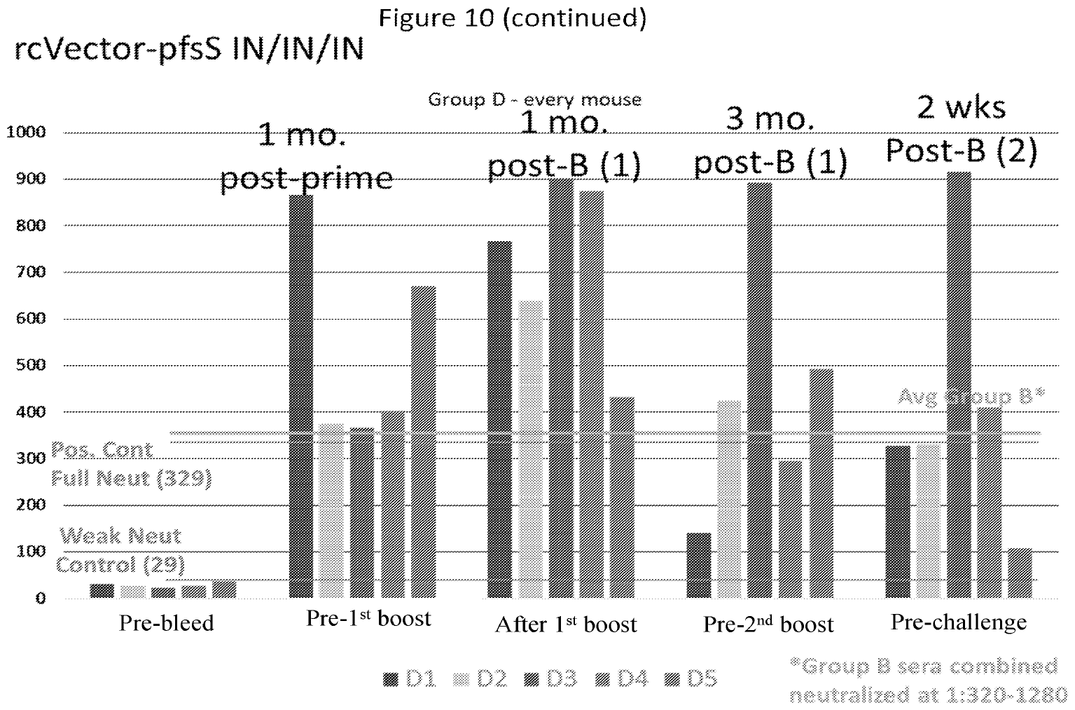

We next tested the ability of our candidate replication-competent vaccine vector to protect mice from challenge with a mouse-adapted SARS-COV-2 virus. Mice in groups A (received 3 sub-cutaneous injections of the vaccine vector), C (received a first dose intranasally, followed by two sub-cutaneous injections of the vaccine vector described here), and D (received three intranasal administration of the vaccine vector) were vaccinated with a prime and two boosts of our candidate replication-competant vaccine, whereas mice in group B were vaccinated with a prime of a plant-derived VLP intramuscularly and two boosts of the replication-competent vaccine sub-cutaneously. All of these mice were protected from viral challenge, as evidenced by the fact that their body weights remained stable for at least 10 days post infection (FIG. 7). In contrast, the mice in groups E-G, which were vaccinated with a prime and two boosts of the VLP or a replication-deficient vaccine vector were not protected and lost weight rapidly (FIG. 7). The clincal scores that were used to determine study endpoints (i.e., when to euthanize the mice) reveal a huge divergence between the outcomes for protected and non-protected mice (FIG. 8). The ability of the replication-competant vaccine to generate SARS-COV-2 neutralizing antibodies was demonstrated by assaying for neutralizing activity in serum samples collected from the group A-D mice at five time points: before prime, one month after prime (prior to the 1st boost), one month after the 1st boost, three months after the 1st boost (prior to the 2nd boost), and two weeks after the 2nd boost (FIG. 10).

### Example 4—Intranasal Immunization with a Vaccinia Virus Vaccine Vector Expressing Pre-Fusion Stabilized SARS-COV-2 Spike Fully Protected Mice Against Lethal Challenge with the Heavily Mutated Mouse-Adapted SARS2-N501YMA30 Strain of SARS-COV-2

The recently identified Omicron variant of SARS-COV-2 has been designated a variant of concern because of its highly mutated spike protein (1). Of particular concern, Omicron spike is mutated at 5 positions (K417, N440, E484, Q493 and N501) that have been associated with escape from neutralizing antibodies induced by either infection with or immunization against the early Washington strain of SARS-COV-2 (see Table 1, SEQ ID NOs: 18-37)(2-4). Thus, Omicron may be able to at least partially escape from immunization with the current vaccines, which are all based on early, unmutated spike proteins.

TABLE 1
RBD Mutations

| | | | | | SARS2- |
|---|---|---|---|---|---|
| | Beta | Gamma | Delta | Omicron | N501Y_{MA30} |
| | (SEQ ID NO) | (SEQ ID NO) | (SEQ ID NO) | (SEQ ID NO) | (SEQ ID NO) |
| G339 | — | — | — | G339D (18) | — |
| S371 | — | — | — | S371L (19) | — |
| S373 | — | — | — | S373P (20) | — |
| S375 | — | — | — | S375F (21) | — |
| K417* | K417N (22) | K417T (23) | — | K417N (22) | K417M (24) |
| N440* | — | — | — | N440K (25) | — |
| G446 | — | — | — | G446S (26) | — |
| L452 | — | — | L452R (27) | — | — |
| S477 | — | — | — | S477N (28) | — |
| T478 | — | — | T478K (29) | T478K (29) | — |
| E484* | E484K (30) | E484K (30) | — | E484A (31) | E484K (30) |
| Q493* | — | — | — | Q493K (32) | Q493R (33) |
| G496 | — | — | — | G4965 (34) | — |
| Q498 | — | — | — | Q498R (35) | Q498R (35) |
| N501* | N501Y (36) | N501Y (36) | — | N501Y (36) | N501Y (36) |
| Y505 | — | — | — | Y505H (37) | — |

*Mutations associated with antibody escape

While the vaccines currently licensed or authorized for emergency use in the United States provide excellent protection against early variants of SARS-COV-2, including Delta, they have limitations that may hinder their widespread worldwide use. They require maintenance of a significant cold-chain, and are administered parenterally, both of which may make widespread use difficult. We have generated a highly attenuated, replication-competent vaccinia virus vector, NYVAC-KC (5), which does not require an extensive cold-chain and can be administered either by scarification on the skin or intranasally (this manuscript). NYVAC-KC is fully replication competent in human primary keratinocytes and primary human dermal fibroblasts (5). Despite being replication competent, NYVAC-KC is highly attenuated in the very sensitive newborn intra-cranial mouse model, as well as in immune-deficient mice (5). NYVAC-KC induced mild induration on the skin of rabbits, with no signs of systemic spread (5). NYVAC-KC was highly immunogenic, inducing improved T cell and antibody responses to HIV inserts, compared to its replication deficient parental vector, NYVAC (5-10). Thus, NYVAC-KC may have properties that will make it useful in the worldwide fight against SARS-COV-2

In this manuscript we describe protection against challenge with a mouse-adapted variant of SARS-COV-2, SARS2-N501YMA30 (11). Early strains of SARS-COV-2 are not pathogenic in mice. SARS2-N501YMA30 was generated by serially passaging through mice of Washington strain SARS-COV-2 that had an N501Y spike mutation. After 30 passages the virus became pathogenic for mice, which was associated with increased affinity for mouse ACE2 protein (11). During passage through mice 4 mutations accumulated in spike (along with 3 mutations in orf1a and 1 non-coding mutation in TRS), K417, E484, Q493, Q498 along with maintenance of the previous mutation at N501. All 5 spike sites mutated in SARS2-N501YMA30 are also mutated in Omicron, and 4 of the 5 mutated sites are at residues which when mutated allow escape from neutralizing antibodies induced by spike from early strains of SARS-COV-2 (2-4). Thus, SARS2-N501YMA30 expresses a highly mutated spike, which may also allow for escape from neutralizing antibodies induced by the current vaccines. However, we show that intranasal immunization with a pre-fusion stabilized Washington strain spike, expressed from the highly attenuated, replication-competent vaccinia virus vector NYVAC-KC, fully protected mice against both death and disease after infection with SARS2-N501YMA30. Immunization by scarification fully protected against death, but not from mild disease. Thus, Washington strain spike, when expressed from a highly attenuated, replication-competent heat-stable poxvirus vector, administered without parenteral injection, can fully protect against challenge with the heavily mutated, mouse-adapted SARS2-N501YMA30 variant of SARS-COV-2.

### Results

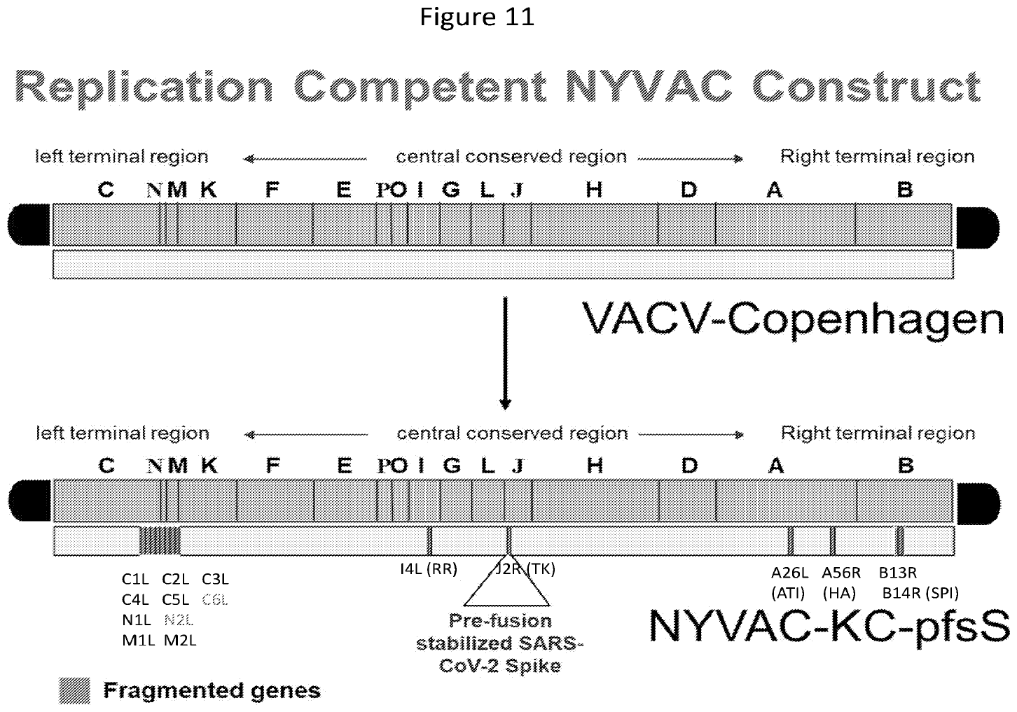

Generation of NYVAC-KC-pfsSpike. A vaccinia virus-optimized Washington strain spike was stabilized in the pre-fusion state by mutation of the furin cleavage site, and insertion of 6 proline residues, preventing the conformational change to the post-fusion conformation (pfsSpike) (12). PfsSpike, flanked by TK locus homologous flanking arms, was inserted into the TK locus of NYVAC-KC by homologous recombination (FIG. 11). The TK locus of NYVAC-KC was modified by insertion of a pGNR-cmr^{S} cassette (13) prior to homologous recombination with TK flanked pfsSpike. pGNR-cmr^{S} encodes a neo^{r} gene and a GFP gene, to allow for selection and identification of virus that has taken up pGNR-cmr^{S}, as well as a cmr^{S} gene that acts as a negative selectable marker (14). Cells were infected with NYVAC-KC-neo^{R}-GFP-cmrS and transfected with TK-flanked pfsSpike. Recombinant virus that had replaced the pGNR-cmrS cassette with pfsSpike was selected for as cmr^{R}, non-fluorescent plaques Insertion of pfsSpike was confirmed by PCR and Western blot of individual plaques. This technology allows for rapid insertion (approximately 1 month from obtaining DNA to having a P2 stock) of new genes into NYVAC-KC.

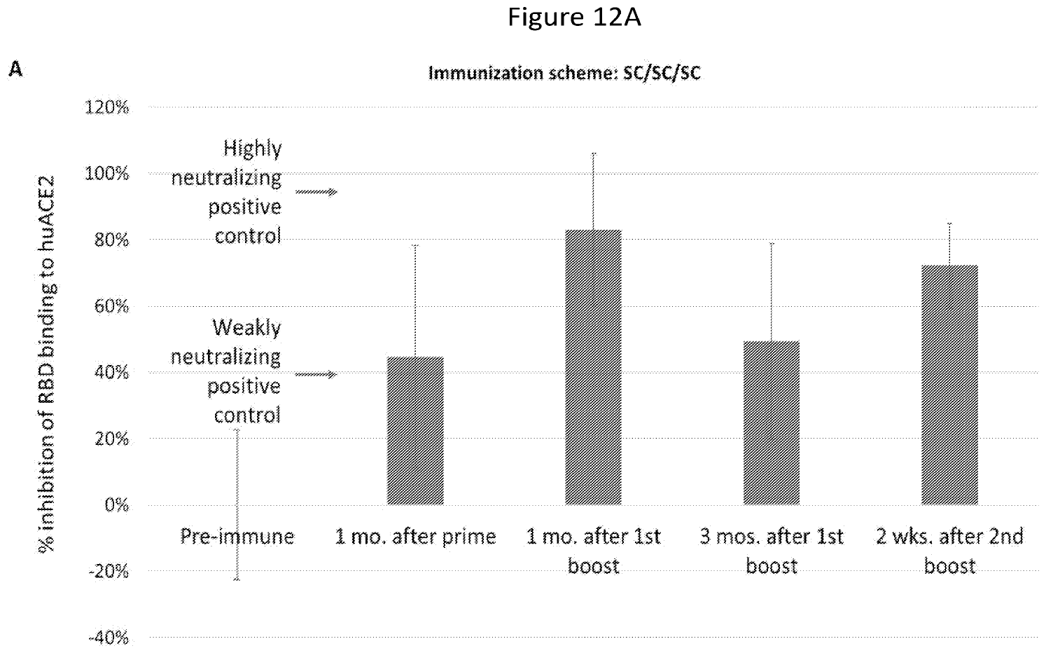

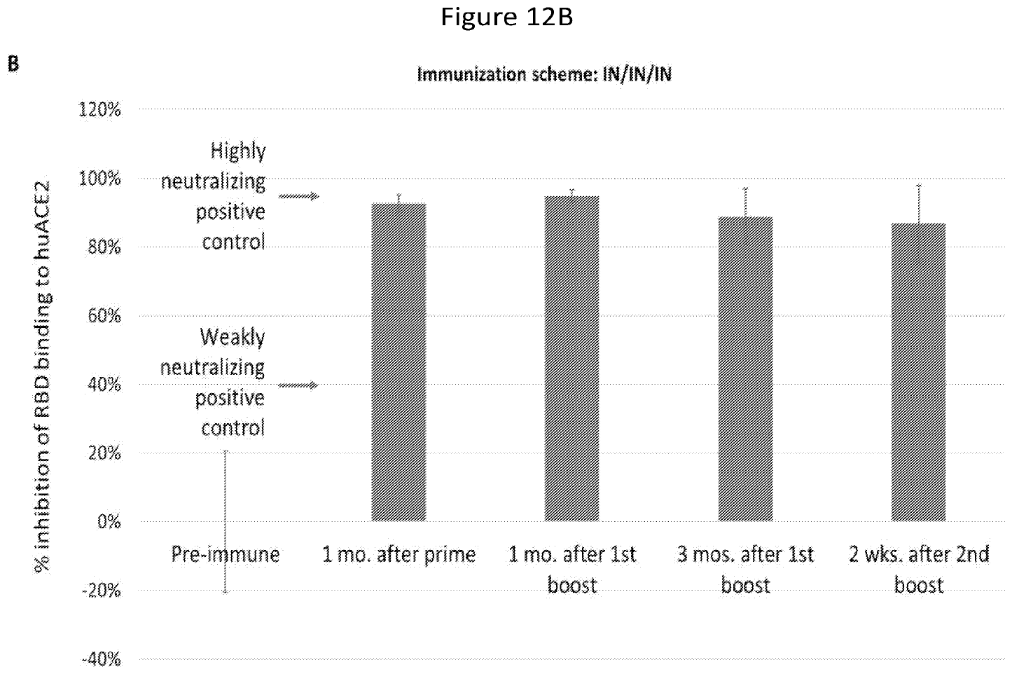

Immunization with NYVAC-KC. Mice were immunized with 100 pfu of NYVAC-KC-pfsSpike, either by scarification or intranasally. Mice were boosted at one month post immunization, rested for 3 months, and boosted a second time. Blood was obtained one month after the primary immunization, one and three months after the first boost and two weeks after the second boost. Serum was assayed for the ability to block binding of Washington strain Spike protein RBD to human ACE2 (15). Immunization by scarification with NYVAC-KC-pfsSpike gave a modest serum response inhibiting RDB binding to huACE2 (FIG. 12A). The response was boosted to high levels, which waned after three months. The second boost increased the serum response, inhibiting binding of RBD to huACE to moderate levels. A single intranasal immunization with NYVAC-KC-pfsSpike induced a potent serum response that inhibited RDB binding to huACE2 (FIG. 12B). This response remained high after the first boost and did not appreciably wane three months after the first boost and remained high after the second boost. Thus, intranasal immunization was able to induce a potent durable serum RBD binding response.

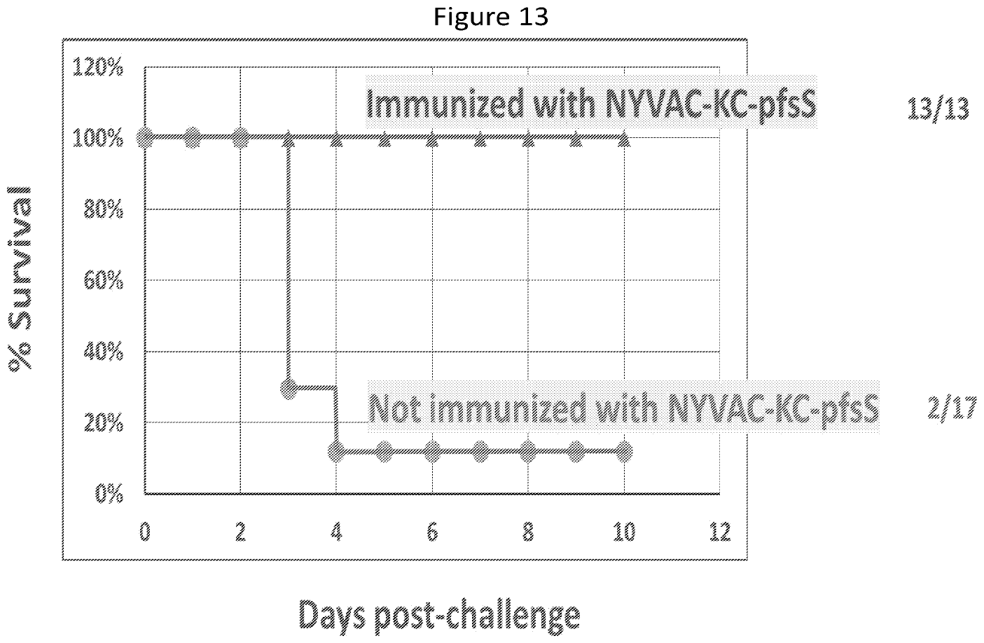

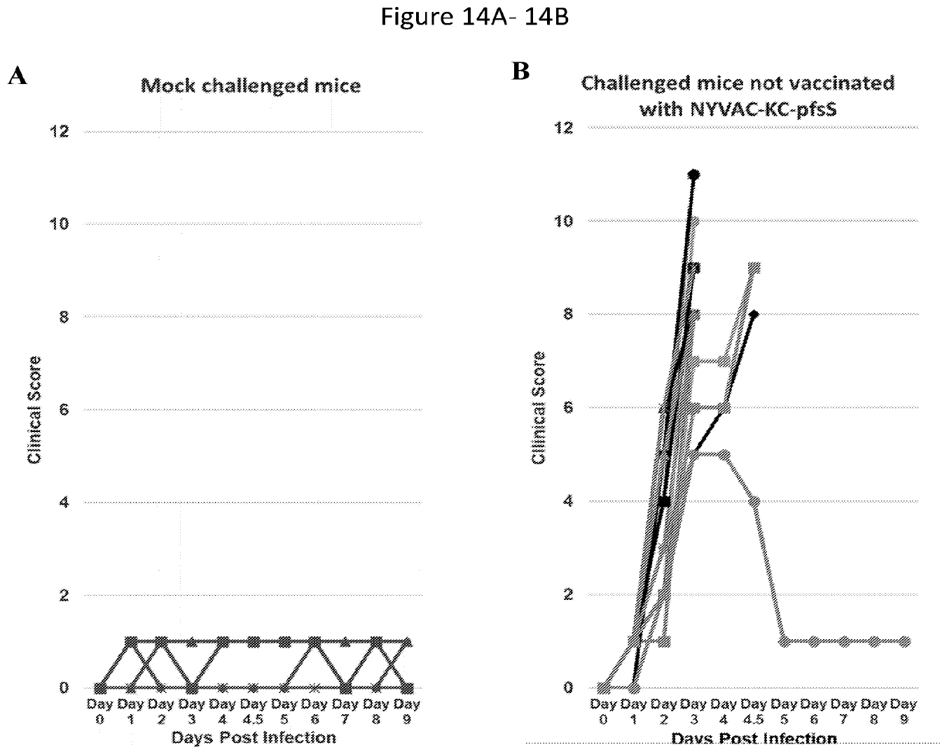

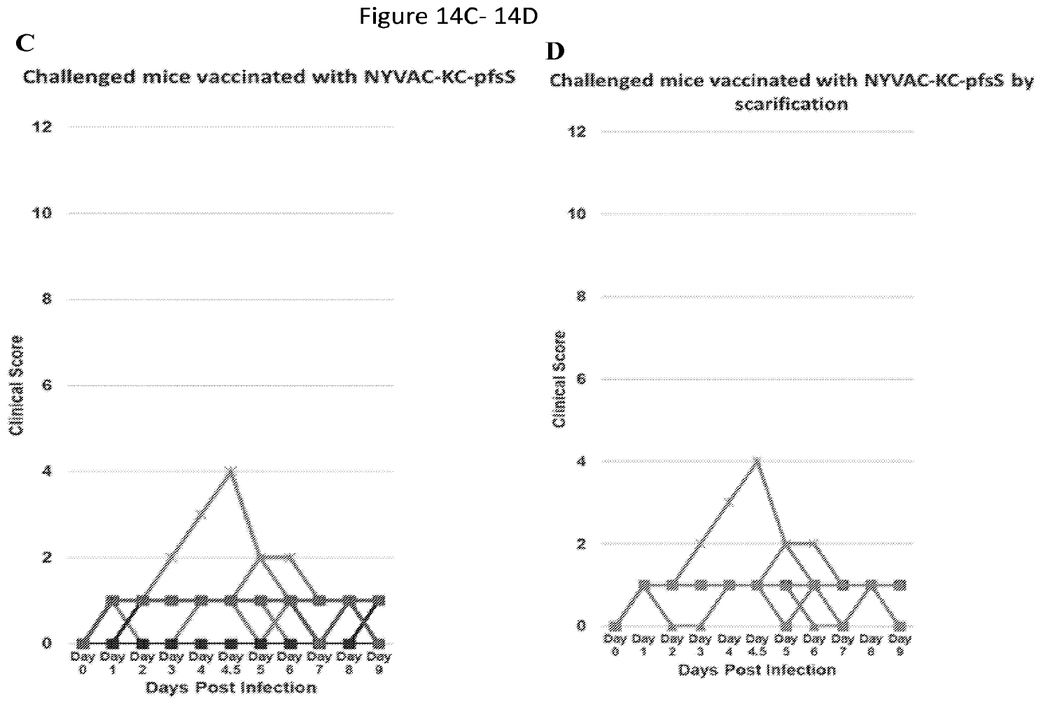

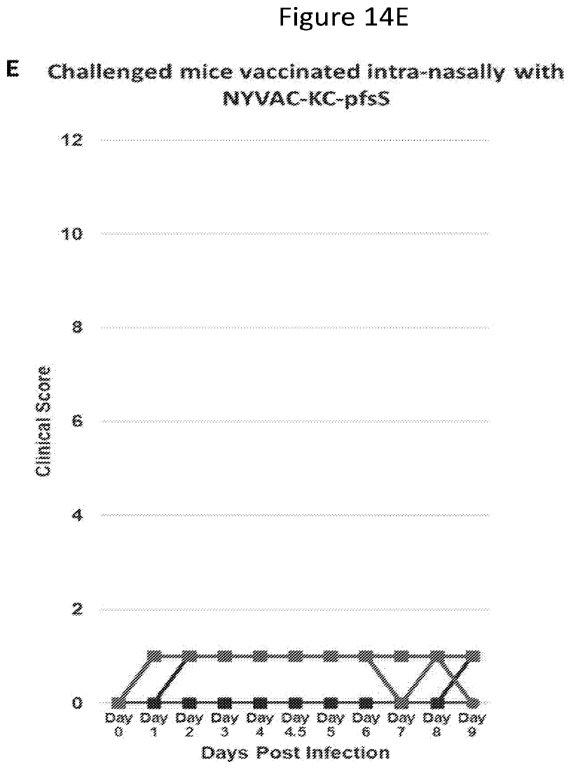

Challenge with SARS2-N501YMA30. Animals immunized with NYVAC-KC-pfsSpike were challenged two weeks after the second boost with approximately 2×10³ pfu of SARS2-N501YMA30 (11). Animals were monitored and scored from 0-3 according to severity for each criterion: weight loss, ruffled fur, hunching, and loss of activity. All animals were scored in a blinded fashion. An aggregate clinical score of 8 was an endpoint for humane euthanasia. Fifteen of seventeen animals not immunized with NYVAC-KC-pfsSpike reached a clinical score of 8 by 4 days post-infection and were humanely euthanized (FIG. 13, red line). None of the animals immunized with NYVAC-KC-pfs-Spike needed to be euthanized (FIG. 13, blue line). FIG. 14 shows the clinical score for each animal in aggregate groups from 0-9 days post challenge. Mock challenged animals had scores of 0-1 throughout the course of the experiment (FIG. 14A). Animals not immunized with NYVAC-KC-pfsSpike, and challenged with SARS2-N501YMA30, all showed signs of illness by days 2-3 post-challenge, and for 15 of the 17 animals, symptoms were serious enough to warrant humane euthanasia (FIG. 14B). Animals immunized with NYVAC-KC-pfsSpike were all spared progression to serious disease (FIG. 14C), although two of the animals had mild disease, with maximal clinical scores of 2 and 4. The two animals that showed mild disease were in the cohort that was immunized by scarification (FIG. 14D). Intranasally immunized animals were asymptomatic after SARS2-N501YMA30challenge, with clinical scores of 0-1 (FIG. 14E), indistinguishable from mock challenged animals (FIG. 14A).

### Materials and Methods

Viruses. Mouse adapted SARS-COV-2 SARS2-N501YMA30 was propagated in A549-huACE2 cells (11). For insertion of foreign genes into the NYVAC-KC genome, we constructed a cassette (pGNR-cmrS) that encodes an E. coli gyrase/PKR fusion protein that confers coumermycin (cmr) sensitivity (14), a neoR gene and expresses GFP (13). The cassette has arms that are homologous to the sequence flanking the TK deletion in NYVAC-KC, to allow for in vivo recombination with the viral genome. The pGNR-cmrS cassette was added to NYVAC-KC through an in vivo recombination (17) done in BSC-40 cells; cells were transfected with linear cassette DNA using Lipofectamine 2000 (Invitrogen) according to product instructions. Infection with NYVAC-KC was at an MOI of 0.05. After 48 hours, the infected cells were scraped into the medium (1.2 mls Opti-Pro (Gibco) with glutamine and 1% FBS). Following two cycles of freeze/thaw, the cell supernatant was used to infect 100 mm dishes of BSC-40 cells, at 1:10, 1:100, and 1:1000 dilutions of the IVR stock. DMEM 2% FBS plus G418 at 1 mg/ml was added after the infection incubation. Green, G418R plaques were picked at 48 hours post infection, following the addition of an agarose overlay. Plaques were screened in 6-well plates for sensitivity to cmr, and the two showing the highest sensitivity were chosen for continuing to the next round of plaque purification in BSC-40 cells. The plaque from this round that demonstrated the highest sensitivity to cmr was amplified in a 60 mm dish. This virus (NYVAC-KC-neoR-GFP-cmrS) was used in an IVR to replace the pGNR-cmrS cassette with the coding sequence for a vaccinia virus optimized, pre-fusion-stabilized SARS-COV-2 Washington strain spike protein (12), under control of a vaccinia virus synthetic early/late promoter (18), yielding a cmrR, non-fluorescent virus. For this selection, 100 ng/ml cmr was added at 24 hpi of the IVR, and subsequent infections were carried out in the presence of cmr until the final plaque was chosen. Correct insertion was confirmed by PCR and Western blotting. Plaques were amplified twice to obtain P2 stocks (5) that were used for immunization of mice.

Cell lines. African green monkey kidney Vero cells (E6) or (CCL81) (obtained from ATCC) were cultured in Dulbecco's modified Eagle's medium (DMEM; Gibco catalog no. 11965), supplemented with 10% fetal bovine serum (FBS), 100 U/ml of penicillin, 100 μg/ml streptomycin, 50 ng/ml gentamicin, 1 mM sodium pyruvate, and 10 mM HEPES. Human A549 cells (Verified by ATCC) were cultured in RPMI 1640 (Gibco catalog no. 11875) supplemented with 10% FBS, 100 U/ml of penicillin, and 100 μg/ml streptomycin. The generation of A549-ACE2 cells was described previously (19).

Plaque assay. Briefly virus supernatant was serially diluted 10-fold and inoculum was absorbed on Vero cells for 1 hour at 37ºC. Inoculum was overlaid with DMEM plus 0.7% agarose and incubated for 3 days at 37ºC. Cells were fixed with 4% paraformaldehyde and stained with 1% crystal violet for counting plaques. All infections and virus manipulations were conducted in a biosafety level 3 (BSL-3) laboratory using appropriate and IBC-approved personal protective equipment and protocols.

Immunization. BALB/c mice at age 7 weeks were immunized with 106 pfu of NYVAC-KC-pfsSpike. Immunization was performed either intranasally (in 10 μL), or by tail scarification (20 μL) and under anesthesia with a cocktail containing 37.5 mg/kg ketamine, 7.5 mg/kg xylazine, and 2.5 mg/kg acepromazine. Following vaccination, mice were allowed to recover on heating pads and were monitored until ambulatory, at which point they were placed in their cages. Mice were boosted 1 month and 4 months after initial vaccination. Throughout the duration of the study before challenge, mice were weighed weekly and blood draws were taken on a bi-weekly basis.

Inhibition of RBD/huACE2 interaction. Neutralizing antibodies were assessed using a lateral flow assay that semi-quantitatively measures levels of antibodies that prevent binding of Washington strain RBD to ACE2, as previously described (15). Briefly, 3 μl of serum was diluted to 6 μl in PBS and loaded onto lateral flow strips that had soluble gold-labeled Washington strain RBD, and bound huACE2. Serum and gold-labeled RBD were chased through the strip with chase buffer (15). After 20 minutes, blue color at the site of the bound ACE2 was quantified by densitometry. Percent inhibition was calculated as previously described (20), using the following formula: 1−(Test sample line density/Limit of Detection, LoD)*100 where LoD for non-neutralizing sera for the rapid test was 570,229.

SARS2-N501YMA30 Challenge. Mice either immunized or not immunized with NYVAC-KC-pfsSpike were moved to the ABSL3 for SARS-COV-2 challenge. SARS2-N501YMA30 was administered intranasally at a dose of 2×10³ pfu per animal in a volume of 50 μl. Mice were anesthetized by intraperitoneal route with a cocktail of 50 mg/kg ketamine and 7.5 mg/kg xylazine for the inoculation. Following the inoculation, mice were allowed to recover in their cages, which were placed on heating pads, and mice were monitored until ambulatory. Mice were weighed daily unless weight fell below 85% of their original weight, at which time they were monitored twice daily. Symptoms were scored in a blinded manner for ruffled fur, hunching and activity, and scored from 0-3 (0 normal, 3 severe) for 10 days and mice were euthanized when their aggregate clinical score reached 8 (including a score of 0-3 for weight loss) as detailed in the approved IACUC protocol. Mice that recovered or were asymptomatic were monitored for 10 days.

### REFERENCES

21. Kibler, K. V., et al., Replication-Competent NYVAC-KC Yields Improved Immunogenicity to HIV-1 Antigens in Rhesus Macaques Compared to Nonreplicating NYVAC. J Virol, 2019. 93(3).
22. Kibler, K. V., et al., Improved NYVAC-based vaccine vectors. PLOS One, 2011. 6(11): p. e25674.
23. Meador, L. R., et al., A heterologous prime-boosting strategy with replicating Vaccinia virus vectors and plant-produced HIV-1 Gag dgp41 virus-like particles. Virology, 2017. 507: p. 242-256.
24. Vijaysri, S., et al., Vaccinia viruses with mutations in the E3L gene as potential replication-competent, attenuated vaccines: intra-nasal vaccination. Vaccine, 2008. 26(5): p. 664-76.
25. Li, F., Structure, Function, and Evolution of Coronavirus Spike Proteins. Annu Rev Virol, 2016. 3(1): p. 237-261.
26. Lu, R., et al., Genomic characterisation and epidemiology of 2019 novel coronavirus: implications for virus origins and receptor binding. Lancet, 2020.
27. Wan, Y., et al., Receptor recognition by novel coronavirus from Wuhan: An analysis based on decade-long structural studies of SARS. J Virol, 2020.
28. Zhou, P., et al., A pneumonia outbreak associated with a new coronavirus of probable bat origin. Nature, 2020.
29. Jiang, L., et al., Potent neutralization of MERS-Col′ by human neutralizing monoclonal antibodies to the viral spike glycoprotein. Sci Transl Med, 2014. 6(234): p. 234ra59.
30. Ying, T., et al., Exceptionally potent neutralization of Middle East respiratory syndrome coronavirus by human monoclonal antibodies. J Virol, 2014. 88(14): p. 7796-805.
31. Tai, W., et al., Recombinant Receptor-Binding Domains of Multiple Middle East Respiratory Syndrome Coronaviruses (MERS-COV's) Induce Cross-Neutralizing Antibodies against Divergent Human and Camel MERS-Cov's and Antibody Escape Mutants. J Virol, 2017. 91(1).
32. Niu, P., et al., Ultrapotent Human Neutralizing Antibody Repertoires Against Middle East Respiratory Syndrome Coronavirus From a Recovered Patient. J Infect Dis, 2018. 218(8): p. 1249-1260.
33. Xiao, X., et al., The SARS-Cov'S glycoprotein: expression and functional characterization. Biochem. Biophys. Res. Commun., 2003. 312(4): p. 1159-1164.
34. Li, W., et al., Angiotensin-converting enzyme 2 is a functional receptor for the SARS coronavirus. Nature, 2003. 426(6965): p. 450-454.
35. Li, F., Receptor recognition mechanisms of coronaviruses: a decade of structural studies. J Virol, 2015. 89(4): p. 1954-64.
76. Graham, R. L., et al., Evaluation of a recombination-resistant coronavirus as a broadly applicable, rapidly implementable vaccine platform. Commun Biol, 2018. 1: p. 179.
77. Ba, L., et al., Heterologous MVA-S prime Ad5-S boost regimen induces high and persistent levels of neutralizing antibody response against SARS coronavirus. Appl Microbiol Biotechnol, 2007. 76(5): p. 1131-6.
78. Volz, A., et al., Protective Efficacy of Recombinant Modified Vaccinia Virus Ankara Delivering Middle East Respiratory Syndrome Coronavirus Spike Glycoprotein. J Virol, 2015. 89(16): p. 8651-6.
79. Wellensiek, B. P., et al., A leader sequence capable of enhancing RNA expression and protein synthesis in mammalian cells. Protein Sci, 2013. 22(10): p. 1392-8.
80. White, S. D., et al., Use of a negative selectable marker for rapid selection of recombinant vaccinia virus. Biotechniques, 2011. 50(5): p. 303-9.
81. Lan, J., et al., Significant Spike-Specific IgG and Neutralizing Antibodies in Mice Induced by a Novel Chimeric Virus-Like Particle Vaccine Candidate for Middle East Respiratory Syndrome Coronavirus. Virol Sin, 2018. 33(5): p. 453-455.
82. Ishii, K., et al., Neutralizing antibody against severe acute respiratory syndrome (SARS)-coronavirus spike is highly effective for the protection of mice in the murine SARS model. Microbiol Immunol, 2009. 53(2): p. 75-82.
83. Al-Allaf, F. A., et al., Modifying inter-cistronic sequence significantly enhances IRES dependent second gene expression in bicistronic vector: Construction of optimised cassette for gene therapy of familial hypercholesterolemia. Noncoding RNA Res, 2019. 4(1): p. 1-14.
84. Koh, E. Y., et al., An internal ribosome entry site (IRES) mutant library for tuning expression level of multiple genes in mammalian cells. PLOS One, 2013. 8(12): p. e82100.
85. Langland, J. O., et al., Products of the porcine group (rotavirus NSP3 gene bind specifically to doublestranded RNA and inhibit activation of the interferon-induced protein kinase PKR. J Virol, 1994. 68(6): p. 3821-9.
86. Li, Y., Self-cleaving fusion tags for recombinant protein production. Biotechnol Lett, 2011. 33(5): p. 869-81.

### Example 4 References

1. WHO. 2021. Classification of Omicron (B.1.1.529): SARS-Cov-2 Variant of Concern.
2. Lu L, Chu A W, Zhang R R, Chan W M, Ip J D, Tsoi HW, Chen L L, Cai J P, Lung D C, Tam A R, Yau Y S, Kwan M Y, To W K, Tsang O T, Lee L L, Yi H, Ip T C, Poon R W, Siu G K, Mok B W, Cheng V C, Chan K H, Yuen K Y, Hung I F, To K K. 2021. The impact of spike N501Y mutation on neutralizing activity and RBD binding of SARS-Cov-2 convalescent serum. EBioMedicine 71:103544.
3. Starr T N, Greaney A J, Addetia A, Hannon W W, Choudhary M C, Dingens A S, Li J Z, Bloom J D. 2021. Prospective mapping of viral mutations that escape antibodies used to treat COVID-19. Science 371:850-854.
4. Weisblum Y, Schmidt F, Zhang F, DaSilva J, Poston D, Lorenzi J C, Muecksch F, Rutkowska M, Hoffmann H H, Michailidis E, Gaebler C, Agudelo M, Cho A, Wang Z, Gazumyan A, Cipolla M, Luchsinger L, Hillyer C D, Caskey M, Robbiani D F, Rice C M, Nussenzweig M C, Hatziioannou T, Bieniasz P D. 2020. Escape from neutralizing antibodies by SARS-Cov-2 spike protein variants. Elife 9.
5. Kibler K V, Gomez C E, Perdiguero B, Wong S, Huynh T, Holechek S, Arndt W, Jimenez V, Gonzalez-Sanz R, Denzler K, Haddad E K, Wagner R, Sekaly R P, Tartaglia J, Pantaleo G, Jacobs B L, Esteban M. 2011. Improved NYVAC-based vaccine vectors. PLOS One 6:e25674.
6. Garcia-Arriaza J, Perdiguero B, Heeney J L, Seaman M S, Montefiori D C, Yates N L, Tomaras G D, Ferrari G, Foulds K E, Roederer M, Self S G, Borate B, Gottardo R, Phogat S, Tartaglia J, Barnett S W, Burke B, Cristillo A D, Weiss D E, Lee C, Kibler K V, Jacobs B L, Wagner R, Ding S, Pantaleo G, Esteban M. 2017. HIV/AIDS Vaccine Candidates Based on Replication-Competent Recombinant Poxvirus NYVAC-C-KC Expressing Trimeric gp140 and Gag-Derived Virus-Like Particles or Lacking the Viral Molecule B19 That Inhibits Type I Interferon Activate Relevant HIV-1-Specific B and T Cell Immune Functions in Nonhuman Primates. J Virol 91.
7. Kibler K V, Asbach B, Perdiguero B, Garcia-Arriaza J, Yates N L, Parks R, Stanfield-Oakley S, Ferrari G, Montefiori D C, Tomaras G D, Roederer M, Foulds K E, Forthal D N, Seaman M S, Self S, Gottardo R, Phogat S, Tartaglia J, Barnett S, Cristillo A D, Weiss D, Galmin L, Ding S, Heeney J L, Esteban M, Wagner R, Pantaleo G, Jacobs B L. 2019. Replication-Competent NYVAC-KC Yields Improved Immunogenicity to HIV-1 Antigens in Rhesus Macaques Compared to Nonreplicating NYVAC. J Virol 93.
8. Perdiguero B, Gomez C E, Cepeda V, Sanchez-Sampedro L, Garcia-Arriaza J, Mejias-Perez E, Jimenez V, Sanchez C, Sorzano C O, Oliveros J C, Delaloye J, Roger T, Calandra T, Asbach B, Wagner R, Kibler K V, Jacobs B L, Pantaleo G, Esteban M. 2015. Virological and immunological characterization of novel NYVAC-based HIV AIDS vaccine candidates expressing clade (trimeric soluble gp140(ZM96) and Gag(ZM96)-Pol-Nef(CN54) as virus-like particles. J Virol 89:970-88.
9. Quakkelaar E D, Redeker A, Haddad E K, Harari A, McCaughey S M, Duhen T, Filali-Mouhim A, Goulet J P, Loof N M, Ossendorp F, Perdiguero B, Heinen P, Gomez C E, Kibler K V, Koelle D M, Sekaly R P, Sallusto F, Lanzavecchia A, Pantaleo G, Esteban M, Tartaglia J, Jacobs B L, Melief C J. 2011. Improved innate and adaptive immunostimulation by genetically modified HIV-1 protein expressing NYVAC vectors. PLOS One 6:e16819.
10. Zurawski G, Shen X, Zurawski S, Tomaras G D, Montefiori D C, Roederer M, Ferrari G, Lacabaratz C, Klucar P, Wang Z, Foulds K E, Kao S F, Yu X, Sato A, Yates N L, LaBranche C, Stanfield-Oakley S, Kibler K, Jacobs B, Salazar A, Self S, Fulp W, Gottardo R, Galmin L, Weiss D, Cristillo A, Pantaleo G, Levy Y. 2017. Superiority in Rhesus Macaques of Targeting HIV-1 Env gp140 to CD40 versus LOX-1 in Combination with Replication-Competent NYVAC-KC for Induction of Env-Specific Antibody and T Cell Responses. J Virol 91.
11. Roy Wong L Y, Zheng J, Wilhelmsen K, Li K, Ortiz M E, Schnicker N J, Pezzulo A A, Szachowicz P J, Klumpp K, Aswad F, Rebo J, Narumiya S, Murakami M, Meyerholz D K, Fortney K, McCray P B, Perlman S. 2021. Eicosanoid signaling as a therapeutic target in middle-aged mice with severe COVID-19. bioRxiv doi: 10.1101/2021.04.20.440676.
12. Hsieh C L, Goldsmith J A, Schaub J M, DiVenere A M, Kuo H C, Javanmardi K, Le K C, Wrapp D, Lee A G, Liu Y, Chou C W, Byrne P O, Hjorth C K, Johnson N V, Ludes-Meyers J, Nguyen A W, Park J, Wang N, Amengor D, Lavinder J J, Ippolito G C, Maynard J A, Finkelstein I J, Mclellan J S. 2020. Structure-based design of prefusion-stabilized SARS-CoV-2 spikes. Science 369:1501-1505.
13. Hansen S G, Cope T A, Hruby D E. 2002. BiZyme: a novel fusion protein-mediating selection of vaccinia virus recombinants by fluorescence and antibiotic resistance. Biotechniques 32:1178, 1180, 1182-7.
14. White S D, Conwell K, Langland J O, Jacobs B L. 2011. Use of a negative selectable marker for rapid selection of recombinant vaccinia virus. Biotechniques 50:303-9.
15. Lake D F, Roeder A J, Kaleta E, Jasbi P, Pfeffer K, Koelbela C, Periasamy S, Kuzmina N, Bukreyev A, Grys T E, Wu L, Mills J R, McAulay K, Gonzalez-Moa M, Seit-Nebi A, Svarovsky S. 2021. Development of a rapid point-of-care test that measures neutralizing antibodies to SARS-Cov-2. J Clin Virol 145:105024.
16. Sartbaeva AaD, A. 2021. The Challenge of Vaccine Cold Chain Logistics: How a Chemical ‘Casing’ Could Save Lives. Global Biodefense.
17. Shors T, Jacobs B L. 1997. Complementation of deletion of the vaccinia virus E3L gene by the Escherichia coli RNase III gene. Virology 227:77-87.
18. Chakrabarti S, Sisler J R, Moss B. 1997. Compact, synthetic, vaccinia virus early late promoter for protein expression. Biotechniques 23:1094-7.
19. Li Y, Renner D M, Comar C E, Whelan J N, Reyes H M, Cardenas-Diaz F L, Truitt R, Tan L H, Dong B, Alysandratos K D, Huang J, Palmer J N, Adappa N D, Kohanski M A, Kotton D N, Silverman R H, Yang W, Morrisey E E, Cohen N A, Weiss S R. 2021. SARS-CoV-2 induces double-stranded RNA-mediated innate immune responses in respiratory epithelial-derived cells and cardiomyocytes. Proc Natl Acad Sci USA 118.
20. Lake D R, A. Gonzalez-Moa, M. Koehler, M. Kaleta, E. Jasbi, P. Vanderhoof, J. Mckechnie, D. Forman, J. Edwards, B. Seit-Nebi, A. Svarovsky, S. 2021. Third COVID-19 Vaccine Dose Boosts Neutralising Antibodies in Poor Responders. MedRxiv doi:https://doi.org/10.1101/2021.11.30.21266716.

### Embodiments of the Invention

Embodiment 1: A recombinant NYVAC vector comprising a promoter operably connected to a translation enhancing element (TEE) and a polynucleotide encoding a severe acute respiratory syndrome coronavirus 2 (SARS-COV-2) antigen; and a polynucleotide encoding C7L (SEQ ID NO:2) adjacent to a polynucleotide encoding KIL (SEQ ID NO:3).

Embodiment 2: The recombinant NYVAC vector of embodiment 1, wherein the SARS-CoV-2 antigen is selected from the group consisting of SEQ ID NO:1, SEQ ID NO:6, SEQ ID NO:7, SEQ ID NO:8, SEQ ID NO:9, or a mutant SARS-COV-2 Spike or RBD of SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, or SEQ ID NO: 37 and sequences at least 90% identical thereto, and combinations thereof.

Embodiment 3: The recombinant NYVAC vector of embodiment 1 or 2, wherein the SARS-COV-2 antigen is ID NO: 1 or a sequence at least 90% identical thereto.

Embodiment 4: The recombinant NYVAC vector of any of embodiments 1-3, wherein the vector comprises polynucleotides encoding at least two SARS-COV-2 antigens selected from the group consisting of SEQ ID NO:1, SEQ ID NO:6, SEQ ID NO:7, SEQ ID NO:8, SEQ ID NO:9, SEQ ID NO: 17 or a mutant SARS-COV-2 Spike or RBD of SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, or SEQ ID NO: 37 and sequences at least 90% identical thereto, and combinations thereof.

Embodiment 5: The recombinant NYVAC vector of any of embodiments 1-4, wherein the vector comprises polynucleotides encoding at least three SARS-COV-2 antigens selected from the group consisting of SEQ ID NO:1, SEQ ID NO:6, SEQ ID NO:7, SEQ ID NO:8, SEQ ID NO:9, SEQ ID NO: 17 or a mutant SARS-COV-2 Spike or RBD of SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, or SEQ ID NO: 37 and sequences at least 90% identical thereto, and combinations thereof.

Embodiment 6: The recombinant NYVAC vector of any of embodiments 1-5, wherein the vector comprises polynucleotides encoding at least four SARS-COV-2 antigens selected from the group consisting of SEQ ID NO:1, SEQ ID NO:6, SEQ ID NO:7, SEQ ID NO:8, SEQ ID NO:9, SEQ ID NO: 17 or a mutant SARS-COV-2 Spike or RBD of SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, or SEQ ID NO: 37 and sequences at least 90% identical thereto, and combinations thereof.

Embodiment 7: The recombinant NYVAC vector of any of embodiment 1-6, additionally comprising an internal ribosomal entry site (IRES).

Embodiment 8: The recombinant NYVAC vector of any of embodiment 1-7, comprising at least 2 IRES.

Embodiment 9: The recombinant NYVAC vector of any of embodiments 1-8, wherein the translation enhancing element comprises SEQ ID NO:4, SEQ ID NO:10, SEQ ID NO:11 or SEQ ID NO:12.

Embodiment 10: The recombinant NYVAC vector of any of embodiments 1-9, additionally comprising a synthetic late promoter (SLP).

Embodiment 11: The recombinant NYVAC vector of embodiment 10, wherein the SLP comprises SEQ ID NO:5.

Embodiment 12: The recombinant NYVAC vector of any of embodiments 1-11, additionally comprising a self-cleaving protein element.

Embodiment 13: The recombinant NYVAC of any of embodiments 1-12, comprising at least two self-cleaving protein elements.

Embodiment 14: A vaccine composition comprising the recombinant NYVAC vector of any of embodiments 1-13 and a pharmaceutically acceptable carrier.

Embodiment 15: The vaccine composition of embodiment 14, additionally comprising an adjuvant.

Embodiment 16: A method of inducing an immune response against a SARS-COV-2 antigen in a subject comprising administering an effective amount of the vaccine composition of embodiment 14 or 15 to the subject.

Embodiment 17: The method of embodiment 16, wherein the SARS-COV-2 antigen is selected from the group consisting of SEQ ID NO:1, SEQ ID NO:6, SEQ ID NO:7, SEQ ID NO:8, SEQ ID NO:9, SEQ ID NO: 17 or a mutant SARS-COV-2 Spike or RBD of SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, or SEQ ID NO: 37 and sequences at least 90% identical thereto, and combinations thereof.

Embodiment 18: The method of embodiments 16 or 17, wherein the SARS-COV-2 antigen is SARS-COV-2 S protein (SEQ ID NO:1), a sequence at least 90% identical thereto or a portion of SEQ ID NO: 1.

Embodiment 19: The method of any of embodiments 16-18, wherein the subject is a human deer, cat, dog, cow, mink, ferret or pig.

Embodiment 20: The method of any of embodiments 16-19, wherein the composition is administered by injection.

Embodiment 21: The method of any of embodiments 16-19, wherein the composition is administered to the subject at least twice.

Embodiment 22: The method of any of embodiments 16-19, wherein the composition is administered to the subject at least three times.

We claim:

1. A recombinant NYVAC vector comprising
a promoter operably connected to a translation enhancing element (TEE) and a polynucleotide encoding a severe acute respiratory syndrome coronavirus 2 (SARS-COV-2) antigen; and
a polynucleotide encoding C7L (SEQ ID NO:2) adjacent to a polynucleotide encoding KIL (SEQ ID NO:3).

2. The recombinant NYVAC vector of claim 1, wherein the SARS-COV-2 antigen is selected from the group consisting of SEQ ID NO:1, SEQ ID NO:6, SEQ ID NO:7, SEQ ID NO:8, SEQ ID NO:9, or a mutant SARS-COV-2 Spike or RBD of SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, or SEQ ID NO: 37 and sequences at least 90% identical thereto, and combinations thereof.

3. The recombinant NYVAC vector of claim 2, wherein the SARS-COV-2 antigen is ID NO:1 or a sequence at least 90% identical thereto.

4. The recombinant NYVAC vector of claim 2, wherein the vector comprises polynucleotides encoding at least two SARS-COV-2 antigens selected from the group consisting of SEQ ID NO:1, SEQ ID NO:6, SEQ ID NO:7, SEQ ID NO:8, SEQ ID NO:9, SEQ ID NO: 17 or a mutant SARS-COV-2 Spike or RBD of SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, or SEQ ID NO: 37 and sequences at least 90% identical thereto, and combinations thereof.

5. The recombinant NYVAC vector of claim 4, wherein the vector comprises polynucleotides encoding at least three SARS-COV-2 antigens selected from the group consisting of SEQ ID NO:1, SEQ ID NO:6, SEQ ID NO:7, SEQ ID NO:8, SEQ ID NO:9, SEQ ID NO: 17 or a mutant SARS-COV-2 Spike or RBD of SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, or SEQ ID NO: 37 and sequences at least 90% identical thereto, and combinations thereof.

6. The recombinant NYVAC vector of claim 5, wherein the vector comprises polynucleotides encoding at least four SARS-COV-2 antigens selected from the group consisting of SEQ ID NO:1, SEQ ID NO:6, SEQ ID NO:7, SEQ ID NO:8, SEQ ID NO:9, SEQ ID NO: 17 or a mutant SARS-COV-2 Spike or RBD of SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, or SEQ ID NO: 37 and sequences at least 90% identical thereto, and combinations thereof.

7. The recombinant NYVAC vector or claims 4-6, additionally comprising an internal ribosomal entry site (IRES).

8. The recombinant NYVAC vector of claims 5-6, comprising at least 2 IRES.

9. The recombinant NYVAC vector of claim 1, wherein the translation enhancing element comprises SEQ ID NO:4, SEQ ID NO: 10, SEQ ID NO: 11 or SEQ ID NO:12.

10. The recombinant NYVAC vector of claim 1, additionally comprising a synthetic late promoter (SLP).

11. The recombinant NYVAC vector of claim 10, wherein the SLP comprises SEQ ID NO:5.

12. The recombinant NYVAC vector of claim 1, additionally comprising a self-cleaving protein element.

13. The recombinant NYVAC of claim 12, comprising at least two self-cleaving protein elements.

14. A vaccine composition comprising the recombinant NYVAC vector of claim 1 and a pharmaceutically acceptable carrier.

15. The vaccine composition of claim 14, additionally comprising an adjuvant.

16. A method of inducing an immune response against a SARS-COV-2 antigen in a subject comprising administering an effective amount of the composition of claim 14 to the subject.

17. The method of claim 16, wherein the SARS-COV-2 antigen is selected from the group consisting of SEQ ID NO:1, SEQ ID NO:6, SEQ ID NO:7, SEQ ID NO:8, SEQ ID NO:9, SEQ ID NO: 17 or a mutant SARS-COV-2 Spike or RBD of SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, or SEQ ID NO: 37 and sequences at least 90% identical thereto, and combinations thereof.

18. The method of claim 17, wherein the SARS-COV-2 antigen is SARS-COV-2 S protein (SEQ ID NO:1), a sequence at least 90% identical thereto or a portion of SEQ ID NO: 1.

19. The method of claim 16, wherein the subject is a human, deer, cat, dog, cow, mink, ferret or pig.

20. The method of claim 16, wherein the composition is administered by injection, oral or intranasal administration.

21. The method of claim 16, wherein the composition of claim 14 is administered to the subject at least twice.

22. The method of claim 21, wherein the composition of claim 14 is administered to the subject at three times.
